(19) United States
(12) Patent Application Publication
(10) Pub. No.: US 2007/0204353 A1
(43) Pub. Date: Aug. 30, 2007

(54) Transgenic animals and methods of monitoring hedgehog responding cells

(76) Inventors: Alexandra Joyner, Larchmont, NY (US); Sohyun Ahn, Rockville, MD (US)

Correspondence Address: KLAUBER & JACKSON, 411 HACKENSACK AVENUE, HACKENSACK, NJ, 07601, US

(21) Appl. No.: 11/509,813

(22) Filed: Aug. 24, 2006

Related U.S. Application Data
(60) Provisional application No. 60/711,047, filed on Aug. 24, 2005.

(51) Int. Cl. A01K 67/027

(52) U.S. Cl. 800/14; 800/18; 800/15; 800/16; 800/17; 800/19

(57) ABSTRACT

Transgenic non-human animal models are described that allow for the fate mapping and identification of cells that respond to hedgehog signaling in vivo during a particular time period. More particularly, these animal models and cells obtained from these animal models may be used to screen for agonists or antagonists of the Hedgehog signaling pathway. Thus, these transgenic animal models allow for the identification of HH agonists or agents that may enhance neurogenesis or for HH antagonists or agents that may be potentially useful for treating cancers or hyperproliferative conditions associated with high levels of hedgehog expression.

### CROSS-REFERENCE TO RELATED APPLICATIONS

The present application is a non-provisional application claiming the priority of copending provisional application Ser. No. 60/711,047, filed Aug. 24, 2005, the disclosure of which is incorporated by reference herein in its entirety. Applicants claim the benefits of this application under 35 U.S.C. §119 (e).

### GOVERNMENT RIGHTS CLAUSE

The research leading to the present invention was supported by National Institutes of Health Grant No. 2R01 1HD35768-06. Accordingly, the Government has certain rights in the invention.

### FIELD OF THE INVENTION

The present invention relates to transgenic non-human animals and cells obtained from these animals, which are useful for monitoring hedgehog responding cells in vivo and in vitro. More specifically, the present invention relates to the use of these animals and cells derived from these animals for genetic fate mapping studies. The present invention provides insight as to the cell types that respond to hedgehog signaling in vivo and in vitro and better delineates how one may develop novel therapeutic strategies for treating neurodegenerative diseases or injuries to the nervous system or for treating cancers or hyperproliferative disorders through the identification of either agonists or antagonists of hedgehog signaling.

### BACKGROUND OF THE INVENTION

Neurogenesis in the adult mammalian brain takes place in the striatal subventricular zone (SVZ) of the lateral ventricular walls of the forebrain and in the subgranular zone (SGZ) of the dentate gyrus of the hippocampus (Lois and Alvarez Buylla, (1994), Science 264:1145-1148; as reviewed in Temple and Alvarez-Buylla, (1999), Curr Opin Neurobiol 9:135-141; Doetsch, F. (2003), Nat. Neurosci 6: 1127-1134; Alvarez-Buylla, et al. (2002), J. Neurosci. 22: 629-634; Gage, F. H. et al. (1998), J. Neurobiol 36: 249-266; Alvarez-Buylla, A. et al. (2004), Neuron 41: 683-686). In these areas or niches there is the persistence of conditions favorable for the existence of stem cells and the generation of new neurons from them. In particular, astrocytes (B cells) function as stem cells in the adult SVZ and generate transiently amplifying cells (C cells) that then differentiate into migrating neuroblasts (A cells) (Doetsch et al., Cell 97:703-716 (1999)). Neuroblasts, i.e., A cells, will then join the rostral migratory stream to reach their final destination in the olfactory bulb where they will terminally differentiate as interneurons (Luskin, Neuron 11(1):173-89 (1993); Lois and Alvarez Buylla, Science 264:1145-1148 (1994)).

However, the mechanisms involved in the orderly production of new neurons from neural stem cells are not clear. During embryogenesis, several secreted signals have been shown to particulate in brain development (Kilpatrick et al., Mol. Cell. Neurosci. 6:2-15 (1996); Temple and Qian, Neuron 15:249-252 (1995); Gritti et al., J. Neurosci. 16:1091-1100 (1996); Li et al., J. Neurosci. 18:8853-8862 (1998); Marbie et al., J. Neurosci. 19:7077-7088 (1999); and Li and LoTurco, Dev. Neurosci. 22:68-73 (2000)). One of these secreted signals is Sonic hedgehog (SHH). SHH is involved in different aspects of development of the early CNS, where it appears to play an important role in cellular specification, differentiation and cell proliferation. For example, SHH is required for the differentiation of floor plate cells and ventral neurons in the early neural tube (Echelard et al., Cell 75:1417-1430 (1993); Krauss et al., Cell 75:1431-1444 (1993); Roelink et al., Cell 76:761-775 (1994); Ruiz i Altaba et al., Mol. Cell. Neurosci. 6:106-121 (1995)) and it is also involved in granule cell precursor proliferation in the cerebellum (Dahmane and Ruiz i Altaba, Development 126:3089-3100 (1999); Wallace, Curr Biol 9:445-448 (1999); Weschler-Reya and Scott, Neuron 22:103-114 (1999); Corrales, et al., Development, 131(22):5581-90, (2004); Lewis, P. M. et al., Dev Biol., 15;270(2):393-410, (2004)). Similarly, SHH promotes the production of neuronal and oligodendroglial lineages in vitro (Kalyani et al., J. Neurosci. 18:7856-7868 (1998); Zhu et al., Dev. Biol. 215:118-129 (1999)); and in vivo (Pringle et al., Dev. Biol. 177:30-42 (1996); Poncet et al., Mech. Dev. 60:13-32 (1996); Orentas et al., Development 126:2419-2429 (1999); Rowitch et al., J Neurosci (1999), 19:8954-8965 (1999); Lu et al., (2000), Neuron 25:317-329).

In the striatal subventricular zone (SVZ) of post-natal and adult mouse brains, stem cell astrocytes give rise to committed neuronal precursors, which then produce neurons. As indicated above, SHH is secreted from precise locations and at defined periods in the embryonic CNS, and has been shown to play an important role in neurogenesis. However, heretofore, the role of SHH had been believed to be solely as a stimulus for committed neuronal precursor cell proliferation or as an inductive signal for embryonic neural tube precursors to differentiate as neurons (see e.g., Ericson et al., Cell 87:661-673 (1996)). For example, SHH and FGF8 or other factors are thought to induce dopaminergic neurons from the anterior neural tube (Ye et al., Cell, 93:755-766 (1998); Matsuura et al., J. Neurosci. 21:4326-4335 (2001)), but this occurs with rarity in vitro (Stull and lacuitti, Exp. Neurol. 169:36-43 (2001)). Indeed, the factor(s) required for the orderly differentiation of adult stem cells into neurons has heretofore not been identified.

While these previous studies demonstrated that SHH is both required for normal proliferation in the SVZ and SGZ and can increase proliferation in these regions, it has not yet been determined whether SHH acts on quiescent neuronal stem cells (NSCs), which are type B, or fast dividing precursor cells, which are type C (Lai, K. et al. (2003), Nat Neurosci 6:21-27; Machold, R. et al. (2003), Neuron, 39: 937-950).

Another area where hedgehog signaling plays a role in disease progression is cancer. For example, prostate cancer is the second most common malignancy in men and the second leading cause of cancer-related death in the United States. It is estimated that more than 30,000 men will die from metastatic prostate cancer this year. Benign prostatic hyperplasia (BPH) is the most common benign neoplasm in men, affecting ˜50% of men over the age of 60. Although BPH does not progress to cancer, the symptoms can significantly diminish daily activities. Furthermore, surgery is often required to alleviate the symptoms, but can cause additional problems. As the population ages, both diseases contribute significantly to national healthcare costs.

The mainstay treatment for advanced prostate cancer is androgen withdrawal therapy, since androgens are required both to maintain existing prostate cancer cells and to promote their growth. Unfortunately, this therapy is only effective for a short time, until non-androgen dependent tumors arise, ultimately leading to metastasis and death. It is becoming apparent that many of the same molecular signaling pathways that function in embryonic development are important in regulating adult organ specific stem cells and inappropriate activation of the pathways that lead to cancer, of which Shh is one linked to prostate cancer. To understand the molecular underpinnings of cancer initiation and metastasis, it is critically important to understand normal development of an organ, as well as the biology of normal adult stem cells and their counterparts in cancer.

Therefore, there is a need to develop a means of identifying cells that respond to SHH, in particular, in neuronal stem cells, as well as in cancer cells. More particularly, there is a need to provide a means of identifying, developing and assessing new therapies that enhance neurogenesis in order to treat subjects suffering from diseases, injuries or other conditions whereby an increase in neurogenesis is desirable. In addition, there is a need for identifying, developing and assessing new therapies that decrease tumor cell growth or to prevent metastasis in order to treat subjects suffering from cancer or other hyperproliferative conditions whereby a decrease in cell growth is desirable. The animal model and cells derived from this animal model described herein may be used to address these needs.

All publications, patent applications, patents and other reference material mentioned are incorporated by reference in their entirety. In addition, the materials, methods and examples are only illustrative and are not intended to be limiting. The citation of references herein shall not be construed as an admission that such is prior art to the present invention.

### SUMMARY OF THE INVENTION

In its broadest aspect, the present invention provides for transgenic non-human animals whose genome comprises a nucleic acid including the key transcriptional regulatory sequences of the Gli1 gene, wherein the transgenic animals serve as models that allow for fate mapping of cells that respond to hedgehog (HH), in particular, sonic hedgehog (Shh), signaling.

In one embodiment, the transgenic non-human animal is a mammal. In another particular embodiment, the animal is a vertebrate. In a more particular embodiment, the transgenic non-human animal is a rodent. In yet another more particular embodiment, the transgenic non-human animal is a mouse. Other examples of transgenic animals include non-human primates, sheep, dogs, pigs, cows, goats, and rabbits. Methods for providing transgenic rabbits are described in Marian et al. (1999) J. Clin. Invest. 104:1683 1692 and James et al. (2000) Circulation 101:1715 1721, herein incorporated by reference in their entirety.

In another particular embodiment, the transgenic non-human animal is a transgenic mouse, Gli1-GFP, whose genome comprises a transgene comprising a nucleic acid sequence encoding a reporter gene operably linked to a Gli1 promoter that directs expression of the reporter gene in a cell of the transgenic animal and its progeny during development and in the adult animal, wherein the reporter gene is only expressed in cells that respond to hedgehog (HH) signaling. This allows for sorting of cells responding to one or more members of the family of hedgehog proteins, for example, sonic hedgehog, based on GFP expression. In another particular embodiment, the nucleic acid sequence encoding a reporter gene operably linked to a Gli1 promoter directs expression of the reporter gene in at least one cell or cell type of the transgenic animal and its progeny during any or all stages of development and in the adult animal.

In another embodiment, the cells from these mice may be isolated based on reporter gene expression and used in microarray studies to identify genes induced by HH. In another particular embodiment, these mice may be used to determine cells of a particular type that respond to HH, for example, stem cells.

In another particular embodiment, the reporter gene is a fluorescent protein selected from the group consisting of, for example, blue, cyan, green, yellow, and red fluorescent proteins.

In another particular embodiment, the transgenic animal is a transgenic mouse, Gli1-CreER^{T2}, which contains a site specific inducible recombinase operably linked to a Gli1 promoter with enhancers and a reporter gene operably linked to a promoter that directs its expression in a cell of the transgenic animal and its progeny during development and in the adult animal. The reporter is constructed such that the reporter gene is only expressed in cells that have a functional Cre recombinase. This animal model allows for fate mapping of any cell that responds to Sonic hedgehog (SHH) signaling and may be used particularly to determine which neuronal stem cells (NSCs), eg. quiescent type B cells or fast-dividing precursor type C cells, respond to Sonic hedgehog signaling and what the cells and their derivatives become. In one embodiment, the Gli 1 promoter directs expression of the recombinase in at least one cell or cell type of the transgenic animal capable of responding to the family of Hedgehog proteins, in particular Sonic hedgehog. In another embodiment, the Gli 1 promoter directs expression of the recombinase in all cells of the transgenic animal capable of responding to one or more members of the family of Hedgehog proteins, in particular Sonic hedgehog. Moreover, exposure of the animal to tamoxifen, progesterone or other inducing agents transiently activates the recombinase, and such activation results in the recombination of two loxP or other site specific recombination target sequences, which results in genetic marking of the cells and their progeny.

In one particular embodiment, the Gli1 promoter directs expression of the inducible recombinase in any cell of the transgenic animal that responds to any one or more members of the family of Hedgehog (HH) proteins. In another particular embodiment, the Gli1 promoter directs expression of the inducible recombinase in at least one cell or cell type of the transgenic animal that responds to one or more members of the family of Hedgehog (HH) proteins. In another particular embodiment, the Gli1 promoter directs expression of the inducible recombinase in all cells or cell types of the transgenic animal that respond to one or more members of the family of Hedgehog (HH) proteins.

In another embodiment, the exposure of the animal to an inducing agent, such as but not limited to, tamoxifen or progesterone, activates the recombinase. The activation of the recombinase results in the recombination of site specific recombination target sequences such as two LoxP sites which results in the genetic marking of the cells and their progeny.

In another embodiment, the reporter gene is selected from the group consisting of β-galactosidase (lacZ), luciferase, a green fluorescent protein, a yellow fluorescent protein, a red fluorescent protein, alkaline phosphatase, or retrograde or anterograde transynaptic tracers such as wheat germ agglutinin (WGA), or derivatives thereof, such as a wheat germ agglutinin-fusion molecule including but not limited to WGA-HRP (wheat germ agglutinin-horseradish peroxidase) or tWGA-dsRED (wheat germ agglutinin-discosoma red), GFP-TTC (green fluorescent protein labeled non-toxic C terminal of tetanus toxin) or any molecule that allows for retrograde and/or anterograde trans-synaptic tracing.

In another embodiment, the transgenic non-human animals according to the present invention include without limitation rodents, such as rats, mice, guinea pigs and gerbils, as well as dogs, cats, pigs, sheep, cows, goats, horses, and rabbits. In yet another embodiment, these transgenic animals are used for fate mapping of cells that respond to Hedgehog signaling in vivo.

Another aspect of the invention provides a method for identifying, marking or following a cell that responds to Hedgehog signaling in vivo. In one embodiment, the method comprises the steps of:
a) preparing a transgenic non-human animal whose genome comprises a transgene comprising a nucleic acid sequence encoding a reporter gene operably linked to a Gli1 promoter that directs expression of the reporter gene in a cell of the transgenic animal and its progeny during development and in the adult animal, wherein the reporter gene is only expressed in a cell that responds to hedgehog (HH) signaling;
b) exposing the transgenic animal to a Hedgehog protein; and
c) examining expression of the reporter gene in a cell obtained from the animal;
wherein a cell that responds to Hedgehog signaling and its progeny demonstrate expression of the reporter gene.

In one particular embodiment, the Gli1 promoter directs expression of the reporter gene in at least one cell or cell type of the transgenic animal and its progeny during development and/or in the adult animal. In another embodiment, the reporter gene is selected from the group consisting of blue, cyan, green, yellow, and red fluorescent proteins. In yet another more particular embodiment, the reporter gene is green fluorescent protein.

Another aspect of the invention provides a method for identifying, marking or following cells responding to Hedgehog signaling in vivo. In one embodiment, the method comprises the steps of:
a) preparing a transgenic non-human animal whose genome comprises a site specific recombinase operably linked to a Gli1 promoter and enhancer, wherein the promoter directs the expression of an inducible recombinase in a cell responding to a Hedgehog protein, and a reporter gene operably linked to a promoter that directs expression of the reporter in a cell of the animal, wherein the reporter is only expressed in a cell that has a functional recombinase;
b) administering an inducing agent for the site specific recombinase to the animal in an amount sufficient to induce the recombinase activity, wherein such induction results in activation of the recombinase and subsequent expression of the reporter gene in a Hedgehog responding cell of the animal and its progeny; and
c) examining expression of the reporter gene in the cell;
   wherein a cell that responds to Hedgehog signaling at the time of administration and its progeny demonstrate expression of the reporter gene.

In one embodiment, the recombinase is selected from the group consisting of CreER, CreER^{T}, CreER^{T2}, CrePR, FlpER, FlpPR, FlpePR, FlpeER, FlpeER^{T}, FlpeER^{TZ} and derivatives thereof.

In another embodiment, the inducing agent is tamoxifen or progesterone.

In another embodiment, the Hedgehog responding cell is present in mesodermal tissue or epithelial tissue or neural tissue. In a more specific embodiment, the tissue is selected from the group consisting of brain tissue, spinal cord tissue, prostate tissue, blood, lung tissue, liver tissue, bladder tissue, eye, skin, urogenital tract, genitalia, lacrimal gland, hair, gut, pancreas and tumor tissue.

In yet another embodiment, the cell responding to hedgehog signaling may be selected from an embryonic stem (ES) cell, an adult stem cell, a neural stem cell, an epithelial stem cell, a mesodermal stem cell, a hematopoietic stem cell or a tumor stem cell, and any stem cell line derived from the foregoing.

In a more particular embodiment, the cell may be a quiescent stem cell.

Another aspect of the invention provides a method for identifying, marking or following a cell responding to Hedgehog signaling in vitro.

In one embodiment, the method comprises the steps of:
a) preparing a cell whose genome comprises a reporter gene operably linked to a Gli1 promoter that directs the expression of the reporter gene in a cell and its progeny that respond to hedgehog (HH) signaling; or
b) obtaining a cell from a transgenic animal whose genome comprises a reporter gene operably linked to a Gli1 promoter that directs the expression of the reporter gene in a cell and its progeny that respond to hedgehog (HH) signaling; and
c) exposing the cell to a Hedgehog protein;
d) examining expression of the reporter gene in the cell;
   wherein a cell that responds to Hedgehog signaling and its progeny demonstrates expression of the reporter gene.

In yet another embodiment, the method comprises the steps of:
a) preparing a cell whose genome comprises an inducible recombinase gene operably linked to a Gli1 promoter containing all Gli1 regulatory elements sufficient for expression in any cell that responds to Hedgehog signaling, wherein the promoter directs expression of the recombinase in any cell that responds to Hedgehog, and a reporter gene operably linked to a promoter which directs expression of the reporter gene in the cell, wherein the reporter gene is only expressed in a cell that has a functional recombinase; or
b) obtaining a cell from an animal whose genome comprises an inducible recombinase gene operably linked to a Gli1 promoter and enhancer, wherein the promoter directs expression of the recombinase in any cells responding to a Hedgehog protein, and a reporter gene operably linked to a promoter which directs expression of the reporter gene in a cell from the animal, wherein the reporter is constructed such that the reporter molecule is only expressed in a cell that has a functional recombinase; and
c) exposing the cell from either of step a) or step b) to an inducing agent in an amount sufficient to induce the recombinase activity, wherein said induction results in expression of the reporter gene in any cell that responds to a Hedgehog protein; and
d) measuring expression of the reporter gene in the cell,
wherein a cell that responds to hedgehog signaling demonstrates expression of the reporter gene.

In one embodiment, the inducing agent includes, but is not limited to, tamoxifen or progesterone, and the inducing agent is added in vivo during embryonic development to determine the ultimate fate of developing cells and their progeny in the adult. Tamoxifen may also be administered to the adult to determine the fate of cells responding to Hedgehog, including stem cells in the brain, spinal cord and the epithelial or mesodermal cells of all tissues. The inducing agents may also be added to the cells in vitro and these cells then used for screening for responsiveness to hedgehog signaling. Moreover, these cells may be used for screening for agents that modulate hedgehog signaling; thus, screening may be done to identify agonists or antagonists of hedgehog signaling. These cells may be used to determine what cells that respond to Hedgehog become.

In one embodiment, the cells that respond to hedgehog signaling in vitro may be isolated from mesodermal tissue, epithelial tissue, and neural tissue. In a more particular embodiment, the cells may be isolated from brain tissue, spinal cord tissue, blood, prostate tissue, liver tissue, lung tissue, bladder tissue, tumor tissue or from the eye, the lacrimal gland, from genitalia, skin, hair, gut, pancreas, kidney and from the urogenital tract. In another particular embodiment, the cells may be embryonic or adult stem cells, neural stem cells, hematopoietic stem cells, or tumor stem cells. In yet another particular embodiment, the cells are quiescent stem cells. In yet another particular embodiment, the cells may be obtained from the transgenic non-human animals described herein.

Another aspect of the invention provides a method of screening for a test compound or agent that acts as an agonist or an antagonist of Hedgehog signaling in vivo and to determine what the cells become. In one particular embodiment, the method comprises the steps of:
a) providing a transgenic non-human animal whose genome comprises an inducible recombinase gene operably linked to a Gli1 promoter and enhancer and a reporter gene, wherein the promoter directs expression of the recombinase and the reporter gene in a cell that responds to Hedgehog signaling in the transgenic animal upon exposure of the animal to an inducing agent, wherein the reporter is only expressed in a cell that has a functional recombinase;
b) administering a test compound to the animal of step a);
c) exposing the animal to an inducing agent in an amount sufficient to induce recombinase activity;
d) determining which cells express the reporter gene; and
e) comparing the expression pattern of the reporter gene in a cell of the transgenic animals treated with the test compound to the pattern of the reporter gene expressed in a cell of the transgenic animals not exposed to the test compound,
wherein a test compound is identified as an agonist or antagonist of Hedgehog signaling if the expression pattern of the reporter gene is changed in a cell of the animals administered the test compound compared to the pattern of expression of the reporter gene in a cell of the animals not receiving the test compound.

In one embodiment, the test compound is identified as an antagonist of hedgehog signaling if the level of expression of the reporter gene is lower in cells of the animals administered the test compound compared to the level of expression of the reporter gene in cells of the animals not receiving the test compound.

In another embodiment, a test compound is identified as an agonist of Hedgehog signaling if it induces expression of the reporter gene in new cells (cells that did not previously express the reporter) incubated with the test compound compared to the expression of the reporter gene in cells of the animals not receiving the test compound.

In another embodiment, the testing for agonists or antagonists of hedgehog signaling may be done in vitro using cells obtained from the transgenic animals of the present invention, as described herein. Alternatively, cells may be constructed in vitro in a manner similar to those obtained from the transgenic animals and used for screening for agonists or antagonists of hedgehog signaling.

In another embodiment, the animals or cells used for screening of agonists or antagonists of Hedgehog signaling are prepared as described above and the inducible recombinase may be selected from the group consisting of CreER, CreER^{T}, CreER^{T2}, CrePR, FlpER, FlpPR, FlpePR, FlpeER, FlpeER^{T}, FlpeER^{TZ} and derivatives thereof.

In yet another particular embodiment, the inducing agent for Cre recombinase may be tamoxifen or progesterone. In yet another embodiment, the range of the inducing agent in vivo is about 10 mg/kg to about 500 mg/kg. In a more preferred embodiment, the range of the inducing agent in vivo is about 25 mg/kg to about 250 mg/kg.

In yet another particular embodiment, the tissue from which the cells may be derived for in vitro screening is mesodermal tissue, epithelial tissue or neural tissue. In a more specific embodiment, the tissue is selected from the group consisting of brain tissue, spinal cord tissue, prostate tissue, lung tissue, liver tissue, bladder tissue, tumor tissue, or from the eye, the lacrimal gland, from genitalia, skin, hair, gut, pancreas, kidney and from the urogenital tract.

In yet another embodiment, the cell responding to Hedgehog signaling may be selected from an embryonic stem cell or an adult stem cell. In yet another particular embodiment, the cell responding to Hedgehog signaling may be a neural stem cell, an epithelial stem cell, a mesodermal stem cell, a hematopoietic stem cell, a stromal or mesenchymal stem cell, or a tumor stem cell.

In a more particular embodiment, the cell may be a quiescent stem cell.

In another embodiment, the inducing agent is tamoxifen or progesterone.

The compounds identified as inhibitors/antagonists of SHH signaling can then be used to treat mammals suffering from a cancerous or precancerous condition or mammals having a tumor. These compounds can also be used to prevent tumor or cancer cell growth or proliferation or tumor metastasis in a mammal comprising administering a therapeutically effective amount of a compound that down regulates SHH signaling. Such compounds can also be used to ameliorate symptoms associated with cancer in a mammal. The novel agents identified by the methods described herein may be used alone in the treatment of cancer or a hyperproliferative condition or disorder, or they may be used as adjunct therapy with other agents or anti-cancer drugs to treat these conditions, or they may be used to prevent tumor metastasis, alone or in conjunction with other anti-tumor or anti-metastatic agents. The instant invention also provides for pharmaceutical compositions comprising the antagonists identified using the methods of the present invention.

The compounds identified as agonists of SHH signaling can then be used to treat mammals suffering from neurodegenerative diseases, disorders or injuries to the nervous system, or diseases of the blood, prostate or lung. These compounds can also be used to further treat or diminish neurodegenerative changes in an animal suffering from a neurological disease, disorder or injury. Such compounds can also be used to ameliorate symptoms associated with neurological diseases or disorders in a mammal. Such diseases, disorders or injuries may be selected from the group consisting of spinal cord injury, traumatic brain injury, stroke, Alzheimer's disease, Parkinson's disease, Amyotrophic Lateral Sclerosis (ALS), progressive supranuclear palsy, Creutzfeldt-Jakob Disease, epilepsy, dementia, schizophrenia, and multiple sclerosis. The novel agents identified by the methods described herein may be used alone in the treatment of these diseases, disorders or injuries, or they may be used as adjunct therapy with other agents to treat these conditions. The agents identified by the methods described herein can also be used in cell culture to promote neural stem cell proliferation and/or differentiation in order to obtain enough cells for use in transplantation to the site of neural disease or injury. The instant invention also provides for pharmaceutical compositions comprising the agonists identified using the methods of the present invention. These compositions may be used in vivo or ex vivo.

Other objects and advantages will become apparent from a review of the ensuing detailed description and attendant claims taken in conjunction with the following illustrative drawings. All references cited in the present application are incorporated herein in their entirety.

### BRIEF DESCRIPTION OF THE DRAWINGS

FIG. 1. NSCs respond to Shh and expand over time in vivo. a, Left panel shows a schematic diagram of a sagittal section of adult mouse brain with the neurogenic regions and migratory paths indicated in red lines. Black area indicates the lateral ventricle. The levels of coronal sections shown in panels b-i are indicated by vertical lines. Two right panels show schematics of hemi-coronal sections of adult forebrain at the level of the SVZ and the DG. Confocal images of single optical slices show expression of GFAP (green; NSC) and nlacZ (red; Shh-responding cell) in the SVZ (b) and DG (c) of two-months old Gli1-nlacZ mice. Arrowheads indicate double-labeled cells and the insets show the orthogonal analysis of a representative cell marked in a box. d-i, Genetic fate mapping of Shh-responding cells over time using Gli1-CreER^{T2}; R26R mice. Short-term fate mapping (1 wk) shows no labeled cells in the OB (d) and few cells in the SVZ (e) and DG (h). Six months after marking with tamoxifen (TM), many labeled cells are found in the OB (f), SVZ (g), and DG (i). Red bracket indicates the subgranular zone (SGZ) and black bracket indicates the granular zone (GZ). Scale bars: b,c 20 μm; e-i 50 μm. OB, olfactory bulb; SVZ, subventricular zone; DG, dentate gyrus.

FIG. 2. The quiescent neural stem cells respond to Shh signaling. a, A schematic of the experimental time-course. TM was administered on two consecutive days (arrows) and AraC or saline was infused for one week. Forebrains of operated mice were analyzed at the indicated survival time. b, Left panel shows a schematic of a sagittal section of adult mouse forebrain. Vertical lines indicate the level of coronal sections obtained in panels c-n. Yellow box indicates the area of the lateral wall of the SVZ analyzed for PSA-NCAM (neuroblast marker) immunoreactivity shown in the right panels. One week of AraC treatment effectively abolished the proliferating neuroblasts from the lateral wall of the lateral ventricle compared to the control. Fate-mapped cells were analyzed at the end of (0 d; c-e), one week (g-i), and six months (k-m) after AraC treatment and compared to control treatment at the corresponding time points (f, j, n). Shh-responding cells survived antimitotic treatment of AraC and expanded over time in the SVZ and DG. Arrows indicate the few cells labeled after AraC treatment. Red bracket indicates the subgranular zone (SGZ) and black bracket indicates the granular zone (GZ) of the DG. Scale bars: 50 μm.

FIG. 3. Shh-responding NSCs generate multiple cell types in vivo. Long-term fate mapped cells (one year after TM treatment) were analyzed for co-expression of lacZ (red) and the indicated molecular markers (green). The vast majority of labeled cells are interneurons (NeuN+) in the olfactory bulb (b) and the dentate gyrus (f) as well as few astrocytes (GFAP+) (a, e; arrowheads). c, Fate-mapped neuroblasts (PSA-NCAM+) are found in the rostral migratory stream (RMS). Quiescent NSCs (GFAP+) are found in the SVZ (d) and SGZ (e, arrows). g, h, Few fate-mapped cells in the corpus callosum are oligodendrocytes (arrowheads). Scale bars: a-e and g-h, 10 μm; f, 20 μm.

FIG. 4. Shh-responding neural stem cells are established during late embryogenesis in mice. TM was given on E15.5 and E17.5 to mark cells responding to Shh at E16-17 and E17-18, respectively. One-month-old mouse forebrains show labeled cells in the OB (a, d) and ventral SVZ (b, e) when marked at both time points. Labeled subgranular zone cells of the DG were seen by E17-18 marking (f) but not by E16-17 marking (c) (red bracket and arrowhead). Scale bar, 50 μm.

FIG. 5. Transit-amplifying cells respond to Shh signalling in the SVZ. The initial population of Shh-responding cells was analyzed in both the dorsal-lateral SVZ (a) and ventral SVZ (b), as indicated in the schematic (top left). Olig2 (green; transit-amplifying cells) and lacZ (red; Shh-responding cells) overlap to a greater extent in the dorsal-lateral SVZ (c) than in the ventral SVZ (d). e. Quantification of double-immunostaining results.

FIG. 6. Dlx2+ transit-amplifying cells are greatly diminished one week after AraC treatment in the RMS and SVZ. A schematic of a sagittal section of adult mouse brain is shown in the upper panel with lines indicating the levels of coronal sections taken. Dlx2 (red) and lacZ (green) expression were assessed by double fluorescence RNA in situ analysis. While control treated samples retain robust expression of Dlx2 in the RMS (a) and SVZ (c), AraC treated samples show only a few Dlx2 positive cells (b, d). Insets in panels c and d show dorsal-lateral (dl) region of the SVZ where more Dlx2-expressing cells reside.

FIG. 7. A schematic of the experimental time-course is shown in the upper panel. TM was administered on two consecutive days (arrow) and AraC was infused for one week. Mice were allowed to recover for three months and subjected to a second round of AraC treatment. Forebrains of operated mice were analyzed at the indicated survival time. a, At the end of the second AraC treatment, labeled cells are found in the ventral SVZ (arrows). PSA-NCAM (green) immunoreactivity is abolished while marked cells (red) remain (inset). b, Two weeks after the second AraC treatment, there are more labeled cells in the ventral SVZ (arrows). PSA-NCAM+ cells (green) have repopulated the region (inset).

FIG. 8. Shh-responding NSCs in the SVZ become interneurons of the olfactory bulb. Left schematic diagram shows a hemicoronal section of the olfactory bulb where boxes indicate the regions shown in panels a and d. Fatemapped cells are found in the periglomerular region (a) and granular layer (d). Boxed areas indicate the representative fields of double-immunostaining shown in panels b, d, e, and f. In the periglomerular region, fate-mapped cells (red) extensively co-express GABA (green, b) and TH (green, c), whereas only GABA (e), but not TH (f), overlap significantly in the granular layer. GL, glomerular layer; GrL, granular cell layer.

FIG. 9. NCBI Entrez Gene identification of Gli-Kruppel Family Member GLI1.

FIG. 10. Shows that Gli1-lacZ is expressed in the stroma of adult epithelial organs. Upper left: Prostate; Upper right: Lung; Lower left: Small intestine; Lower right: Colon.

FIG. 11. Shows the expansion of Shh-responding (Gli1-CreER) stromal cells after P14 is dependent on Gli1. Left: Wild type; Right Gli1 Null

FIG. 12. Shows Gli1-CreER marked adult prostate stromal cells expand during regeneration, and are dependent on Gli. Upper left: wild type 1 day after Tamoxifen; Upper right: wild type 14 days after Tamoxifen; Lower left: Gli1 Null 1 day after Tamoxifen; Lower right: Gli1 Null 14 days after Tamoxifen.

### DETAILED DESCRIPTION

Before the present methods and treatment methodology are described, it is to be understood that this invention is not limited to particular methods, and experimental conditions described, as such methods and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated herein by reference.

Definitions

The terms used herein have the meanings recognized and known to those of skill in the art, however, for convenience and completeness, particular terms and their meanings are set forth below.

The term “non-human animal” is used herein to include all animals, except humans. It also includes an individual animal in all stages of development, including embryonic and fetal stages. It includes rodents, including mice, rats, gerbils, etc. It also includes other animals such as goats, sheep, cows, dogs, cats and horses.

A “transgenic animal” is any animal containing one or more cells bearing genetic information altered or received, directly or indirectly, by deliberate genetic manipulation at a subcellular level, such as by targeted recombination or microinjection or infection or transfection with recombinant virus. The term “transgenic animal” is not intended to encompass classical cross-breeding or in vitro fertilization, but rather is meant to encompass animals in which one or more cells are altered by, or receive, a recombinant DNA molecule. This recombinant DNA molecule may be specifically targeted to a defined genetic locus, may be randomly integrated within a chromosome, or it may be extrachromosomally replicating DNA. The term “germ cell line transgenic animal” refers to a transgenic animal in which the genetic alteration or genetic information was introduced into a germ line cell, thereby conferring the ability to transfer the genetic information to offspring. If such offspring in fact possess some or all of that alteration or genetic information, they are transgenic animals as well. Methods for generating transgenic animals via embryo manipulation and microinjection, particularly animals such as mice, have become conventional in the art and are described, for example, in U.S. Pat. Nos. 4,736,866; 4,870,009; 4,873,191; and in Hogan, B., Manipulating the Mouse Embryo, (Cold Spring Harbor Laboratory Press, Cold Spring Harbor, N.Y., 1986), herein incorporated by reference in their entirety. Similar methods are used for production of other transgenic animals. A transgenic animal can be produced by introducing nucleic acid into the male pronuclei of a fertilized oocyte, e.g., by microinjection, retroviral infection, and allowing the oocyte to develop in a pseudopregnant female foster animal. A transgenic founder animal can be identified based upon the presence of the transgene in its genome and/or expression of transgenic mRNA in tissues or cells of the animals. A transgenic founder animal can then be used to breed additional animals carrying the transgene. Moreover, transgenic animals carrying a transgene can further be bred to other transgenic animals carrying other transgenes.

“Transgene” means any piece of DNA which is inserted by artificial means into a cell, animal or organism and becomes part of the genome either by integration or as an extrachromosomal element. “Transgenes” as used herein further means polynucleotides comprising protein coding regions alone or in addition to expression control elements such as internal ribosome entry sequences. A transgene remains in the genome of the mature animal in one or more cell types or tissues of the transgenic animal.

As used herein, the terms “nucleic acid”, “polynucleotide” and “oligonucleotide” refer to primers, probes, and oligomer fragments to be detected, and shall be generic to polydeoxyribonucleotides (containing 2-deoxy-D-ribose), to polyribonucleotides (containing D-ribose), and to any other type of polynucleotide which is an N-glycoside of a purine or pyrimidine base, or modified purine or pyrimidine bases (including abasic sites). There is no intended distinction in length between the term “nucleic acid”, “polynucleotide” and “oligonucleotide”, and these terms will be used interchangeably. These terms refer only to the primary structure of the molecule. Thus, these terms include double- and single-stranded DNA, as well as double- and single-stranded RNA.

A “reporter gene” refers to a gene whose phenotypic expression is easy to monitor and is used to study promoter activity in different tissues or developmental stages. Recombinant DNA constructs are made in which the reporter gene is attached to a promoter region of particular interest and the construct transfected into a cell or organism. As used herein, a “reporter” gene is used interchangeably with the term “marker gene” and is a nucleic acid that is readily detectable and/or encodes a gene product that is readily detectable such as green fluorescent protein (as described in U.S. Pat. No. 5,625,048 issued Apr. 29, 1997, and WO 97/26333, published Jul. 24, 1997, the disclosures of each are hereby incorporated by reference herein in their entireties), or red fluorescent protein, or yellow fluorescent protein, or wheat germ agglutinin (WGA) or a WGA-type molecule or luciferase.

An “agonist”, as used herein, is an endogenous substance or a drug that can interact with a protein in the HH signaling pathway and initiate a physiological or a pharmacological response characteristic of HH proteins, such as induction of Gli1 transcription, or induction of cellular proliferation or differentiation.

An “antagonist” is an endogenous substance or a drug that competes with an agonist or any protein in the HH signaling pathway that blocks the physiological or a pharmacological response characteristic of that signaling pathway. In the present application, an antagonist may block induction of Gli1 transcription or may block cell proliferation or differentiation.

The term “fate mapping” refers to a means by which to trace the development of specific cells of the embryo, or postnatal or adult animal and determine the eventual type of cell they and their progeny will later become. Fate mapping refers to the tracing of cell lineages and following individual cells through development to see what they become.

A “test compound”, “drug candidate”, “candidate compound”, “agent”, or “therapeutic agent” refers to all materials that may be used to prepare pharmaceutical and diagnostic compositions, or that may be compounds such as small synthetic or naturally derived organic compounds, nucleic acids, polypeptides, antibodies, fragments, isoforms, variants, or other materials that may be used independently for such purposes, all in accordance with the present invention. In addition, as related to the present invention, a “test compound”, “drug candidate”, “candidate compound”, “agent”, or “therapeutic agent” relates to any molecule, e.g. a protein or pharmaceutical, i.e., a drug, with the capability of either:

a) substantially inhibiting the growth of a cell for which growth inhibition is desirous, for example, a tumor cell, which has been contacted with said drug candidate, candidate compound, test compound, agent, or therapeutic agent, relative to a tumor cell that has not been contacted with the drug candidate, candidate compound, test compound, agent, or therapeutic agent, or

b) substantially enhancing the proliferation of a stem cell, including, but not limited to, embryonic stem cells, adult stem cells, a neuronal stem cell, a mesodermal stem cell, a mesenchymal or stromal stem cell, or differentiation of a cell, such as, but not limited to a neural stem cell into various cells of the nervous system, including but not limited to neurons, glial cells, oligodendrocytes, or astrocytes.

“Treatment” or “treating” refers to therapy, prevention and prophylaxis and particularly refers to the administration of medicine or the performance of medical procedures with respect to a patient, for either prophylaxis (prevention) or to cure or reduce the extent of or likelihood of occurrence of the infirmity or malady or condition or event in the instance where the patient is afflicted. Most preferably, the treating is for the purpose of reducing or diminishing the symptoms or progression of a disease or disorder.

A “therapeutically effective amount” is an amount sufficient to decrease or prevent the symptoms associated with the conditions disclosed herein.

As used herein, “probe” refers to a labeled oligonucleotide primer, which forms a duplex structure with a sequence in the target nucleic acid, due to complementarity of at least one sequence in the probe with a sequence in the target region. Such probes are useful for identification of a target nucleic acid sequence according to the invention. Pairs of single-stranded DNA primers can be annealed to sequences within a target nucleic acid sequence or can be used to prime DNA synthesis of a target nucleic acid sequence.

Procedures using such conditions of high stringency are as follows. Prehybridization of filters containing DNA is carried out for 15 minutes to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 h at 65° C. in prehybridization mixture containing 100 μg/ml denatured salmon sperm DNA and 5-20×10⁶ cpm of ³²P-labeled probe. Washing of filters is done at 37° C. for 1 h in a solution containing 2×SSC, 0.01% PVP, 0.01% Ficoll, and 0.01% BSA. This is followed by a wash in 0.1×SSC at 50° C. for 45 min before autoradiography. Other conditions of high stringency that may be used are well known in the art.

“Subject” or “patient” refers to a mammal, preferably a human, in need of treatment for a condition, disorder or disease.

A “small molecule” or “small organic molecule” is an organic compound (or organic compound complexed with an inorganic compound (e.g., metal)) that has a molecular weight of less than 3 kilodaltons, and preferably less than 1.5 kilodaltons.

As used herein, the term “Hedgehog (HH) signaling pathway” is the signaling pathway initiated by a Hedgehog protein binding to its receptor leading to the transcription of a Gli mRNA. The factors involved in or that can function in this pathway include any Hedgehog protein such as Sonic hedgehog, the “Sonic hedgehog (SHH) signaling pathway”, or any one of the following: Indian hedgehog and Desert hedgehog, Patched (Ptc) 1 and 2, Smoothened (Smo), Gli1, Gli2, Gli3, agonists and antagonists of such proteins, PKA, Fused, Suppressor of fused, Costal-2, and modifiers and/or partners of any of the Gli 1, 2, or 3 proteins e.g., the Zic gene products.

As used herein, the term “Hedgehog” is used interchangeably with the term “HH” or “Hh” and is a protein that binds to the HH receptor to stimulate the beginning of the hedgehog signaling pathway.

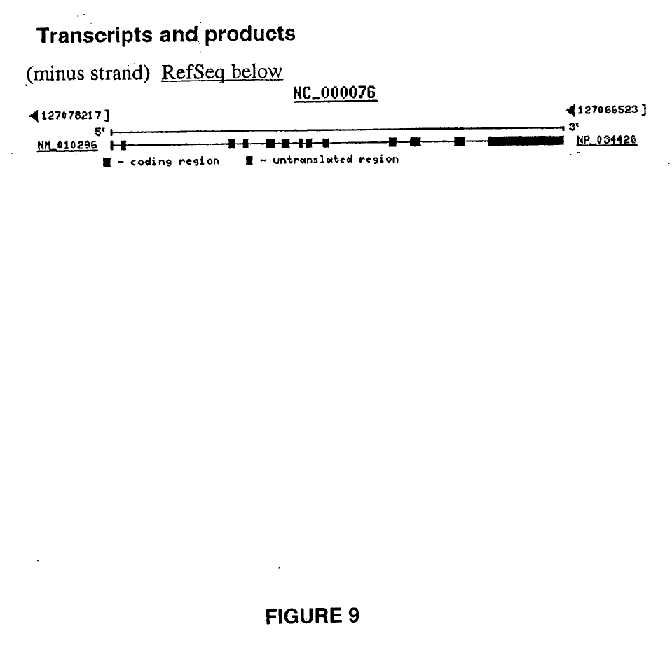

The term “transcriptional regulatory sequence” is intended to include promoters, enhancers and other expression control elements (e.g., polyadenylation signals) or response elements or inducible elements that modulate expression of a nucleic acid sequence. Such regulatory sequences are described, for example, in Goeddel; Gene Expression Technology. Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). Regulatory elements typically do not themselves code for a gene product. Instead, regulatory elements affect expression of the coding sequence, i.e., transcription of the coding sequence. The transcriptional regulatory sequences for the mouse Gli1 gene can be found in PubMed accession numbers NM_{—}010296, NP_{—}034426 and NC_{—}000076, as well in the NCBI Entrez Gene site noted in FIG. 9.

A “promoter” or “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

“Enhancers” or “promoter enhancers” are cis-acting elements of DNA, usually about from 10 to 300 bp, that act on a promoter to increase its transcription. Many enhancer sequences are now known from mammalian genes (globin, elastase, albumin, α-fetoprotein, and insulin). In some cases one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. The enhancer may be spliced into the vector at a position 5′ or 3′ to the coding sequence, but is preferably located at a site 5′ from the promoter. As related to the present invention, the enhancer is a target of HH signaling.

“Operably linked” when describing the relationship between two polynucleotide sequences, means that they are functionally linked to each other. For example, a promoter is operably linked to a coding sequence if it controls the transcription of the sequence. As a regulatory sequence commonly used promoter elements as well as enhancers may be used. Generally, such expression regulation sequences are derived from genes that are expressed primarily in the tissue or cell type chosen. Preferably, the genes from which these expression regulation sequences are obtained are expressed substantially only in the tissue or cell type chosen, although secondary expression in other tissue and/or cell types is acceptable if expression of the recombinant DNA in the transgene in such tissue or cell type is not detrimental to the transgenic animal.

“Quiescent cell” or “quiescent stem cell” refers to a cell or a stem cell that rarely divides, but when it does, it does so indefinitely. It may divide symmetrically to give rise to more quiescent stem cells or it may divide asymmetrically to produce a quiescent cell and a transiently amplifying cell. A population of cells is considered herein to be a population of quiescent stem cells when at least 70%, preferably at least 80%, and more preferably at least 90% of the cells are in the G1 or G0 phase of the cell cycle. Quiescent cells exhibit a single DNA peak by flow-cytometry analysis, a standard technique well known to those of ordinary skill in the arts of immunology and cell biology. Another technique useful for determining whether a population of cells is quiescent is the addition of a chemical agent to the cell culture medium that is toxic only to actively cycling cells, i.e., DNA synthesizing cells, and does not kill quiescent cells. Non-exclusive examples of such chemical agents include hydroxyurea, cytosine arabinoside (AraC) and high specific activity tritiated thymidine. A population of cells is evaluated as to the percent in an actively cycling state by the percent of the cell population killed by the chemical agent. A cell population in which in vitro cell killing is less than approximately 30%, preferably less than approximately 10%, most preferably less than approximately 5%, is considered to be quiescent.

“Transit amplifying cell” or “transient amplifying stem cell” refers to a fast dividing stem cell that has a limited life span, in most cases less than 4 weeks.

“Derivative” refers to either a protein or polypeptide that comprises an amino acid sequence of a parent protein or polypeptide that has been altered by the introduction of amino acid residue substitutions, deletions or additions, or a nucleic acid or nucleotide that has been modified by either introduction of nucleotide substitutions or deletions, additions or mutations. The derivative nucleic acid, nucleotide, protein or polypeptide possesses a similar or identical function as the parent polypeptide, but may have additional properties as well, such as for example, WGA-dsRed, whereby the WGA allows for crossing of the synapse and the dsRed allows for marking or tracing of the molecule.

In a specific embodiment, the term “about” means within 20%, preferably within 10%, and more preferably within 5%.

A “recombinase” refers to an enzyme capable of promoting a reciprocal double-stranded DNA exchange between two DNA segments. Moreover, the term “site-specific” is used to describe a recombinase that recognizes very specific sequences in both partners of the exchange. The target DNA sequences can be on the same or on different strands. If on different strands, an insertion, or integration, as occurs during lysogeny by lambda phage, results. If on the same strand, the outcome depends on alignment of the target sequences. In one alignment, inversion occurs. Examples include events catalyzed in the Flp and Hin inversion systems. In the other alignment, deletion of a circular DNA from a linear DNA, or separation of a circular DNA into two circles results. These reactions occur in the resolution of cointegrates and the resolution of concatemers. The Flp inversion system of the yeast 2 micron plasmid illustrates site-specific recombination catalyzed by an Int-Flp recombinase. The Flp inversion system was discovered on the 2 micron plasmid of S. cerevisiae and consists of a gene coding for a “recombinase” and two flanking “target” sites. The Flp system functions to maintain the number of 2 micron plasmid copies at high levels in cells. Multiple copies result when inversion occurs after a replication fork has passed one of the target sites. Moreover, two site-specific recombinases that are used frequently for engineering the mouse genome include Cre from P1 phage and Flp from yeast. Both enzymes catalyze recombination between two 34-base pair recognition sites, lox and FRT, respectively, resulting in excision, inversion, or translocation of DNA sequences depending upon the location and the orientation of the recognition sites.

General Description

The present invention relates to the use of a Genetic Inducible Fate Mapping (GIFM) (Joyner and Zervas, 2006, Developmental Dynamics, In Press) approach to mark and follow cells (including stem cells) responding to hedgehog signaling, in particular, sonic hedgehog (Shh) signaling, in vivo. The studies provided herein demonstrate that Shh signals to both quiescent stem cells and transiently amplifying cells in the adult forebrain. Moreover, this technique is also useful for determining whether Shh also regulates dormant stem cells in epithelial tissues, with an emphasis on the prostate since it is a site of significant human pathology for which the biology is poorly understood (benign prostate hyperplasia and prostate carcinoma). In addition, prostate stem cells can be manipulated in vivo through castration and multiple rounds of androgen driven regeneration. It was determined that Gli1 is required downstream of Shh for expansion of the stromal population ie. likely stem cells, during development and following involution/regeneration. In contrast, using this technique, it was determined that mouse prostate carcinomas have an abnormal induction of Gli1 in epithelial cells.

More particularly, the present invention provides for newly developed transgenic mouse models that enable the analysis and elucidation of cells responding to hedgehog signaling, and which serve as powerful models for lineage tracing or fate mapping of cells responding to hedgehog (HH). Furthermore, the invention provides for the isolation of cells from these animals that may be used for in vitro studies to screen for agonists or antagonists of HH signaling. One may use these animals or cells derived from these animals for evaluating the efficacy of mechanism-based therapeutic agents targeting this signaling pathway. It may be possible to identify certain agents that are agonists of hedgehog (HH) signaling, thus allowing for the identification of novel agents that enhance, for example, neurogenesis. These agents may be useful for the treatment of a number of neurodegenerative diseases or conditions, for which there are currently few, if any, known therapies. The transgenic mice of the present invention, or the cells derived from these mice may also be useful for the identification and development of antagonists of HH signaling, thus identifying potential treatments for certain tumor cells that are regulated by HH signaling.

In one particular embodiment, the present invention provides a transgenic animal, in particular, a mouse, Gli1-eGFP, which contains a reporter gene operably linked to a Gli1 promoter that directs expression of the reporter gene in a cell of the transgenic mouse (and its progeny) that respond to hedgehog (HH) signaling. This allows for sorting of cells responding to, for example, sonic hedgehog, based on GFP expression. In another particular embodiment, the Gli1 promoter directs the expression of the reporter gene in at least one cell or cell type of the animal that responds to hedgehog signaling during any one or in all stages of development and in the adult animal. In another particular embodiment, the Gli1 promoter directs the expression of the reporter gene in all cells of the animal that respond to hedgehog signaling during any one or in all stages of development and in the adult animal.

In another particular embodiment, the invention provides another transgenic non-human animal whose genome comprises a nucleic acid including the key transcriptional regulatory sequences of the Gli1 gene, wherein the transgenic animal serves as a model that allows for fate mapping of cells that respond to sonic hedgehog (SHH) signaling. More particularly, the transgenic animal is a mammal. In another particular embodiment, the mammal is a transgenic mouse, Gli1-CreER^{T2}, which contains a site specific inducible recombinase operably linked to a Gli 1 promoter and enhancer and a reporter gene operably linked to a promoter that directs its expression in at least one hedgehog responding cell of the transgenic animal and its progeny at any one stage of development, or at all times during development and in the adult animal. In another particular embodiment, the transgenic mouse, Gli1-CreER^{T2}, contains a site specific inducible recombinase that is operably linked to a Gli 1 promoter and enhancer and a reporter gene operably linked to a promoter that directs its expression in all hedgehog responding cells of the transgenic animal and their progeny at any one time during development, or at all times during development and in the adult animal. The reporter is constructed such that the reporter molecule is only expressed in cells that have a functional Cre recombinase. This animal model allows for fate mapping of cells that respond to sonic hedgehog (SHH) signaling and may be used particularly to determine which neuronal stem cells (NSCs), eg. quiescent type B cells or fast-dividing precursor type C cells, are responsive to sonic hedgehog signaling determining what they become under different conditions. The Gli 1 promoter enhancer directs expression of the inducible recombinase in all cells of the transgenic animal capable of responding to the family of hedgehog proteins, in particular sonic hedgehog. Moreover, exposure of the animal to tamoxifen, progesterone or other inducing agents transiently activates the recombinase, and such activation results in the recombination of two loxP sites, or other site specific recombination target sequences which results in genetic marking of the cells and their progeny.

Another aspect of the present invention provides a method for identifying various therapeutically active agents including but not limited to proteins, peptides, peptidomimetic drugs, small molecule drugs, chemicals and nucleic acid based agents using these transgenic animals or cells derived from these animals. The novel therapeutic agents may be hedgehog (HH) agonists to be used for treating neurodegenerative diseases or conditions, or for treating injuries of the nervous system, including but not limited to spinal cord injury or traumatic brain injury. In addition, the novel therapeutics may be hedgehog (HH) antagonists to be used for treating cancers or hyperproliferative disorders or pre-cancerous conditions, or malignant cancers or tumors.

The present invention is based on experiments performed in accordance with the present invention, which demonstrates for the first time the stem cells that respond to hedgehog signaling. More particularly, the present invention provides for animal models that allow for fate mapping of cells that respond to sonic hedgehog (SHH) signaling and may be used particularly to determine which neuronal stem cells (NSCs), eg. quiescent type B cells or fast-dividing precursor type C cells, or adult stem cells, are responsive to sonic hedgehog signaling and what they become in different conditions.

The present invention further provides for identifying cells of the developing and regenerating prostate that respond to Shh and that require Gli1. Testing has been done to determine whether Shh has a general function in adult stem cells. In addition, studies have been done to determine whether Shh signals to stem cells in diverse adult tissues. More particularly, to further explore the existence of stromal stem cells in adult epithelial tissues, studies were done on the prostate because of the high incidence of prostate cancer and BPH, and because Shh is required for expansion of embryonic stroma. In addition, the prostate offers a distinct advantage for studying dormant adult stem cells, in that it can be induced to undergo involution and regeneration by castration and subsequent administration of Androgens. Furthermore, involution/regeneration can be induced at least 20 times by withdrawing and administering Androgens.

As an initial approach to determine whether a dormant stromal stem cell population exists in the adult prostate, studies were done using Genetic Inducible Fate Mapping (GIFM) to follow the fate of Shh-responding stromal cells during regeneration following involution caused by castration. This approach also allowed for testing whether rare epithelial stem cells are marked by Gli1-CreER, since epithelial stem cells proliferate during regeneration. Such rare cells would therefore expand and produce extensive lacZ+ marking of the prostate epithelium.

In one of the transgenic animal models, the Gli-CreER; R26 mouse, the Gli 1 promoter enhancer directs expression of the recombinase in a cell of the transgenic animal that responds to the family of hedgehog proteins, in particular sonic hedgehog. Moreover, exposure of the animal to tamoxifen, progesterone or other inducing agents transiently activates the recombinase, and such activation results in the recombination of two loxP sites, or other site specific recombination target sequences, which results in genetic marking of the cells and their progeny.

While the present invention provides for the production of transgenic mouse models, it is understood by those skilled in the art that the present invention, in general, provides for other non-human transgenic animal models including, but not limited to, other rodents, such as rats, guinea pigs and gerbils, as well as, dogs, cats, pigs, sheep, cows, goats, horses, and rabbits. Such transgenic models provide for the identification of the role of HH during neurogenesis, neural growth and development and also to the understanding of the function of HH signaling as involved in other tissues, such as prostate, lung, blood or in tumorigenesis, as well as other conditions in which the gene is responsible and/or related for the testing of possible therapies.

Methods for Introducing Nucleic Acid into an Embryo

Several methods are known in the art to introduce a recombinant nucleic acid molecule into an embryo of a non-human animal. These include, for example, microinjection into a nucleus of a fertilized ovum, retroviral transfection of embryonal cells, and transfection of embryonic stem cells.

Microinjection is a particular method for transforming a zygote or early stage embryo. In the mouse, the male pronucleus reaches the size of approximately 20 micrometers in diameter which allows reproducible injection of 1-2 pl of DNA solution. The use of zygotes as a target for gene transfer has a major advantage in that in most cases the injected DNA will be incorporated into the host gene before the first cleavage (Brinster et al., Proc. Natl. Acad. Sci. USA 82 (1985), 4438-4442). As a consequence, all cells of the transgenic non-human animal will carry the incorporated transgene. This will, in general, also be reflected in the efficient transmission of the transgene to offspring of the founder since 50% of the germ cells will harbor the transgene. Once the DNA molecule has been injected into the fertilized egg cell, the cell is implanted into the oviduct of a recipient female, and allowed to develop into an animal.

Viral infection can also be used to introduce a transgene into a non-human animal e.g., retroviral, adenoviral or any other RNA or DNA viral vectors using known techniques. The developing non-human embryo can be cultured in vitro to the blastocyst stage. During this time, the blastomeres can be targets for retroviral infection (Jaenisch, Proc. Natl. Acad. Sci USA 73: 1260-1264, (1976)). Efficient infection of the blastomeres is obtained by enzymatic treatment to remove the zona pellucida (Hogan, et al. (1986) In Manipulating the Mouse Embryo, Cold Spring Harbor Laboratory Press, Cold Spring Harbor, N.Y.). The viral vector system used to introduce the transgene is typically a replication-defective retrovirus carrying the transgene (Jahner et al., Proc. Natl. Acad. Sci. USA 82: 6927-6931, (1985); Van der Putten et al., Proc. Natl. Acad. Sci. USA 82: 6148-6152, (1985)). Transfection is easily and efficiently obtained by culturing the blastomeres on a monolayer of virus-producing cells (Van der Putten, supra; Stewart et al., EMBO J. 6: 383-388, (1987)).

Another method for introduction of transgenes into a non-human animal is referred to as gene trapping (Gossler, A., Joyner, A. L., Rossant, J., Skames, W. D. (1989), Science, 244: 463-465; Skames et al., (1992), Genes Dev., 6: 903-18; Durick et al., (1999), Genome Res., 9:1019-1025; Pruitt et al. (1992), Development 116:573-583;). The basic gene trap vector contains a splice acceptor site immediately upstream of a reporter gene and a selectable marker. When a gene trap vector integrates into an intron of a gene, the reporter is situated such that it becomes under the transcriptional control of the trapped gene's promoter, the trapped gene being defined by integration of the reporter into the trapped gene's intron. When the trapped gene is transcribed, a fusion transcript is generated between upstream exons and the reporter gene.

More recently, embryonic stem (ES) cells have been employed to generate transgenic animals. Embryonic stem cells are derived from the inner cell mass (ICM) of blastocysts and are totipotent cells which are capable of developing into all cell lineages, including germ cells, when introduced into an embryo. As used herein, “embryo” includes developmental stages wherein ES cells are injected into diploid blastocysts or aggregated with morulae. ES cells are obtained from pre-implantation embryos cultured in vitro (Evans et al., Nature 292: 154-156, (1981); Bradley et al., Nature 309: 255-258, (1984); Gossler et al., Proc. Natl. Acad. Sci USA 83: 9065-9069, (1986); and Robertson et al., Nature 322: 445-448, (1986)). Transgenes can be efficiently introduced into ES cells using a number of means well known to those of skill in the art. Such transformed ES cells can thereafter be combined with blastocysts from a non-human animal. The ES cells thereafter colonize the embryo and contribute to the germ line of the resulting chimeric animal; for a review see Jaenisch, Science 240: 1468-1474, (1988)).

ES cells isolated from blastocysts can be established as permanent cell lines, wherein they can be genetically manipulated. In view of this ability, they constitute an effective tool for modifying the mammalian and particularly the mouse genome by being introduced into the animals, for example, by means of controlled mutations or other genetic modifications. For examples of methods for producing transgenic animals, see U.S. Pat. No. 6,492,575 to Wagner, et al. for “Method for developing transgenic mice”, the disclosure of which is incorporated herein by reference. The isolation of ES cells from blastocysts, the establishing of ES cell lines and their subsequent cultivation are carried out by conventional methods as described, for example, by Doetchmann et al., (1985) J. Embryol. Exp. Morph. 87: 27-45; Li et al., (1992) Cell 69: 915-926. The cultivation of ES cells can be carried out using methods such as those described in Donovan et al (1997, Transgenic Animals, Generation and Use, Ed. L. Houdebine, pp 179-187, Harwood Academic Publishers). As noted above, transgenes can be efficiently introduced into the ES cells using standard methods known from the literature for in vitro transfer of DNA into mammalian cells, such as electroporation (Matise, M., Auerbach, W. and Joyner, A. L. (2000). Production of targeted embryonic stem cell clones. In: Gene Targeting: A Practical Approach, 2^{nd} edition, ed. A. L. Joyner, Oxford University Press, Oxford).

Once transfected, the DNA integrates by homologous recombination into the ES genome. Accordingly, in one embodiment of the present invention provides ES cells that have homologously recombined with a vector of the present invention. Such ES cells can thereafter be combined with blastocysts from a nonhuman animal. This produces a chimeric embryo, which constitutes a mixture of the two embryos. These embryos are then transferred into a pseudo-pregnant mouse which acts as a foster mother. The chimeric offspring obtained have cells, including germ line cells, which originate from one of the two original embryos. In this way, a genetic modification introduced in ES cells by homologous recombination can be introduced in the germ line of a chimeric mouse and be transmitted to generate progeny that are heterozygous for the transgene.

Transgenic Animals

Transgenic animals according to the present invention include without limitation rodents, such as rats, mice, guinea pigs and gerbils. Other transgenic animals envisioned for use as described herein include dogs, cats, pigs, sheep, cows, goats, horses, and rabbits. Transgenic animals are those which have incorporated a foreign gene into their genome. A transgene is a foreign gene or recombinant nucleic acid construct which has been incorporated into a transgenic animal. By breeding and inbreeding such animals, it is possible to produce heterozygous and homozygous transgenic animals.

The success rate for producing transgenic animals is greatest in mice. A number of other transgenic animals have also been produced. These include rabbits, sheep, cattle, rats and pigs (Jaenisch (1988), Hammer et al., J. Animal Sci. 63: 269, (1986); Hammer et al., Nature 315: 680, (1985); Wagner et al., Theriogenology 21: 29, (1984), (1984)). Effective generation of transgenic pigs and mice are also described in, for example, Chang et al., BMC Biotechnol. 2 (1): 5 (2002). Generation of transgenic rabbits is described in James et al., J. Mol. Cell Cardiol. 34: 873-882, (2002), and Murakami et al., Theriogenology 57: 2237-2245, (2002). Furthermore, the generation of transgenic sheep is described for example in Kadokawa et al., Domest. Anim. Endocrinol. 24: 219-229, (2003), and Campbell, Methods Mol. Biol. 180: 289-301, (2002). U.S. Pat. No. 5,639,457 is also incorporated herein by reference to supplement the present teaching regarding transgenic pig and rabbit production. U.S. Pat. Nos. 5,175,384; 5,175,385; 5,530,179, 5,625,125, 5,612,486 and 5,565,186 are also each incorporated herein by reference to similarly supplement the present teaching regarding transgenic mouse and rat production.

Screening for the Transgene in the Transgenic Animal

Screening the offspring of an animal for the expression of a desired transgene can be done by several methods known in the art. For example, RT-PCR can be employed to amplify the transgene or fragments thereof from RNA obtained from the animal, usually from tail clippings or blood. Standard PCR methods useful in the present invention are described in PCR Protocols: A Guide to Methods and Applications (Innis et at., eds., Academic Press, San Diego 1990). It is also possible to introduce a marker together with the transgene. Other techniques include protein based assays, such as ELISA, FISH, RNA in situ hybridization or Western blot techniques, which usually require an antibody directed against an epitope of the transgene or the marker. FISH techniques are described in e.g., Gall et al., Meth. Enzymol., 21: 470-480, (1981), and Angerer et al. in Genetic Engineering: Principles and Methods Setlow and Hollaender, Eds. Vol 7, pgs 43-65, Plenum Press, New York, 1985).

The present invention further relates to a cell line established from the transgenic animal of invention that is capable of responding to HH signaling in vitro. Such a cell line can be easily obtained by methods well known in the art. Short Protocols in Cell Biology (2003, edited by Bonifacino, Dasso, Harford, Lippincott-Schwartz and Yamada, John Wiley & Sons, Inc.) provides a collection of protocols for establishing and maintaining cell lines. In a preferred embodiment the cells are derived from the transgenic animal according to the invention, most preferably from the mouse model described herein.

Instead of propagating the transgenic cells in vitro they can also be transferred to another animal. The transferred cells or tissues will usually be from the donor animal and will respond to HH signaling as described herein. General methods for the transfer of cells and/or tissues from donor onto host animals as well as screening assays related thereupon are known in the art, see, e.g., DE 196 37 645 and WO00/40082. The disclosure content of these references is incorporated herein in their entirety and can be adapted to the embodiments of the present invention. However, tissue size, cell number and transfer procedures may have to be optimized for each donor and host animal.

Similarly, cells and/or tissues can be cultured before being transplanted. Thus, a further embodiment of the present invention provides an animal model for identifying agonists or antagonists of HH signaling and thus may be used to identify agonists useful for treating neurodegenerative diseases or disorders or injuries to the nervous system, or for identifying antagonists to HH signaling such that these agents can be used to treat cancers or hyperproliferative conditions or disorders.

One of skilled in the art would recognize that there are a number of approaches to the use of these transgenic animals for the design and screening of drugs. One approach involves the screening of drug candidates for the prevention or treatment of a tumor disorder comprising transplanting cells from a cell line of the invention into an animal; administering one or more drug candidates to the animal; and evaluating the effect of the drug candidate(s) on the transplanted cells. Another approach involves the screening of drug candidates for the enhancement of neurogenesis comprising transplanting cells from a cell line of the invention into an animal; administering one or more drug candidates to the animal; and evaluating the effect of the drug candidate(s) on the transplanted cells.

In one particular embodiment, a method of screening for a test compound that acts as an agonist or antagonist of Hedgehog signaling in vivo comprises the following steps:
a) providing a transgenic non-human animal whose genome comprises either:
   i. a transgene comprising a nucleic acid sequence encoding an inducible recombinase gene operably linked to a Gli1 promoter enhancer and a reporter gene, activated by the recombinase, wherein the promoter directs expression of the recombinase and the reporter gene in a cell that responds to Hedgehog signaling in the non-human transgenic animal upon exposure of the animal to an inducing agent; or
   ii. a transgene comprising a nucleic acid sequence encoding a reporter gene operably linked to a Gli1 promoter that directs expression of the reporter gene in a cell of the transgenic animal and its progeny that respond to hedgehog signaling during development and in the adult animal, wherein the reporter gene is only expressed in a cell that responds to hedgehog (HH) signaling;
b) administering a test compound to the animal of step a) i or a) ii;
c) exposing the animal to an inducing agent in an amount sufficient to induce recombinase activity if the animal from step a) i is used;
d) observing for a cell and its progeny that expresses the reporter gene; and
e) comparing the level of expression of the reporter gene in animals treated with the test compound to the level of expression of the reporter gene expressed in the transgenic animals not exposed to the test compound,
wherein a test compound is identified as an agonist or antagonist of Hedgehog signaling if the level of expression of the reporter gene is changed in animals administered the test compound compared to the level of expression of the reporter gene in animals not receiving the test compound.

The reporter gene may be selected from the group consisting of β-galactosidase (lacZ), luciferase, a green fluorescent protein, a yellow fluorescent protein, a red fluorescent protein, alkaline phosphatase, or retrograde or anterograde transynaptic tracers such as wheat germ agglutinin (WGA), or derivatives thereof, such as a wheat germ agglutinin-fusion molecule including but not limited to WGA-HRP (wheat germ agglutinin-horseradish peroxidase) or tWGA-dsRED (wheat germ agglutinin-discosoma red), GFP-TTC (green fluorescent protein labeled non-toxic C terminal of tetanus toxin) or any molecule that allows for retrograde and/or anterograde trans-synaptic tracing.

The test compound is identified as an antagonist of Hedgehog signaling if the level of expression of the reporter gene is lower in animals administered the test compound compared to the level of expression of the reporter gene in animals not receiving the test compound.

The test compound is identified as an agonist of Hedgehog signaling if it induces higher expression of the reporter gene in new (not pre-existing cells) cells incubated with the test compound compared to the expression of the reporter gene in mammals not receiving the test compound.

In another particular embodiment, a method is provided for screening for a test compound that acts as an agonist or antagonist of Hedgehog signaling in vitro, wherein the method comprises the following steps:
a) preparing a hedgehog responsive cell whose genome comprises either:
   i. a nucleic acid sequence encoding a Gli1 gene, an inducible recombinase gene and a reporter gene activated by the recombinase, all of which are operably linked to a Gli1 promoter enhancer, wherein the promoter directs expression of Gli1, the recombinase and the reporter gene upon exposure of the cell to an inducing agent; or
   ii. a nucleic acid sequence encoding a reporter gene operably linked to a Gli1 promoter that directs expression of the reporter gene in the cell, wherein the reporter gene is only expressed in a cell that responds to hedgehog (HH) signaling;
b) incubating the cell of step a) i or a) ii. with a test compound;
c) exposing the cell to an inducing agent if the cell of step a) i is used;
d) measuring expression of the reporter gene in the cell and its progeny; and
e) comparing the level of expression of the reporter gene in the cell treated with the test compound to the level of expression of the reporter gene in the cell not exposed to the test compound,
wherein a test compound is identified as an agonist or antagonist of Hedgehog signaling if the level of expression of the reporter gene is changed in the cell treated with the test compound compared to the level of expression of the reporter gene in the cell not treated with the test compound.

The test compound is identified as an antagonist of Hedgehog signaling if the level of expression of the reporter gene is lower in the cell incubated with the test compound compared to the level of expression of the reporter gene in the cell not incubated with the test compound.

The test compound is identified as an agonist of Hedgehog signaling if it induces a higher level of expression of the reporter gene in a cell incubated with the test compound compared to the expression of the reporter gene in a cell not incubated with the test compound.

In another embodiment, the number of cells expressing the reporter gene may be greater in a cell culture system after exposure of the cells to a test compound that is an agonist of HH than in a cell culture system not exposed to the test compound. Likewise, the number of cells expressing the reporter gene may be fewer in a cell culture system after exposure of the cells to a test compound that is an antagonist of HH than in a cell culture system not exposed to the test compound. It is envisioned that the cells may be obtained from the transgenic animals described herein , or they may be prepared using the genetic constructs described herein.

A “test compound”, “drug candidate”, “candidate compound”, “agent”, or “therapeutic agent” refers to all materials that may be used to prepare pharmaceutical and diagnostic compositions, or that may be compounds such as small synthetic or naturally derived organic compounds, nucleic acids, polypeptides, antibodies, fragments, isoforms, variants, or other materials that may be used independently for such purposes, all in accordance with the present invention. In addition, as related to the present invention, a “test compound”, “drug candidate”, “candidate compound”, “agent”, or “therapeutic agent” as used herein, means any molecule, e.g. a protein or pharmaceutical, i.e., a drug, with the capability of either:
a) substantially inhibiting the growth of a cell for which growth inhibition is desirous, for example, a tumor cell, which has been contacted with said drug candidate, candidate compound, test compound, agent, or therapeutic agent, relative to a tumor cell that has not been contacted with the drug candidate, candidate compound, test compound, agent, or therapeutic agent, or
b) substantially enhancing the proliferation of a stem cell, including, but not limited to, an embryonic stem cell, an adult stem cell, a neuronal stem cell, a mesodermal stem cell, a mesenchymal or stromal stem cell, or differentiation of a cell, such as, but not limited to a neural stem cell into various cells of the nervous system, including but not limited to neurons, glial cells, oligodendrocytes, or astrocytes.
   Reporter Genes and Trans-synaptic Tracers

Any reporter transgene can be used in the present invention. In one embodiment, a fluorescent protein is used as the reporter. Any fluorescent protein can be used. For example, green fluorescent proteins (“GFPs”), yellow fluorescent proteins and red fluorescent proteins are suitable fluorescent proteins for use in the fluorescent indicators. Also included as reporter genes or proteins are alkaline phosphatase and horseradish peroxidase.

The isolated nucleic acid molecule encoding a green fluorescent protein can be deoxyribonucleic acid (DNA) or ribonucleic acid (RNA, including messenger RNA or mRNA), genomic or recombinant, biologically isolated or synthetic. The DNA molecule can be a cDNA molecule, which is a DNA copy of a messenger RNA (mRNA) encoding the GFP. In one embodiment, the GFP can be from Aequorea victoria (Prasher et al., “Primary Structure of the Aequorea Victoria Green-Fluorescent Protein,” Gene 111(2):229 233 (1992); U.S. Pat. No. 5,491,084 to Chalfie et al., which are hereby incorporated by reference in their entirety). A plasmid encoding the GFP of Aequorea victoria is available from the ATCC as Accession No. 75547. Mutated forms of GFP that emit more strongly than the native protein, as well as forms of GFP amenable to stable translation in higher vertebrates, are commercially available from Clontech Laboratories, Inc. (Palo Alto, Calif.) and can be used for the same purpose. The plasmid designated pTα1-GFPh (ATCC Accession No. 98299) includes a humanized form of GFP. Indeed, any nucleic acid molecule encoding a fluorescent form of GFP can be used in accordance with the subject invention. Standard techniques are then used to place the nucleic acid molecule encoding GFP under the control of the chosen cell specific promoter.

Several light-generating protein coding sequences are commercially available, including, but not limited to, the following. Clontech (Palo Alto, Calif.) provides coding sequences for luciferase and a variety of fluorescent proteins, including, blue, cyan, green, yellow, and red fluorescent proteins. Enhanced green fluorescent protein (EGFP) variants are well expressed in mammalian systems and tend to exhibit brighter fluorescence than wild-type GFP. Enhanced fluorescent proteins include enhanced green fluorescent protein (EGFP), enhanced cyan fluorescent protein (ECFP), and enhanced yellow fluorescent protein (EYFP). Further, Clontech provides destabilized enhanced fluorescent proteins (dEFP) variants that feature rapid turnover rates. The shorter half life of the dEFP variants makes them useful in kinetic studies and as quantitative reporters. DsRed coding sequences are available from Clontech. DsRed is a red fluorescent protein useful in expression studies. Further, Fradkov, A. F., et. al., described a novel fluorescent protein from Discosoma coral and its mutants which possesses a unique far-red fluorescence (FEB S Lett. 479 (3), 127 130(2000)) (mRNA sequence, GENBANK Accession No. AF27271 1, protein sequence, GENBANK Accession No. AAG16224). Promega (Madison, Wis.) also provides coding sequences for fire fly luciferase. Further, coding sequences for a number of fluorescent proteins are available from GENBANK, for example, accession numbers AY015995, AF322221, AF080431, AF292560, AF292559, AF292558, AF292557, AF139645, U47298, U47297, AY015988, AY015994, and AF292556.

GFPs have also been isolated from the sea pansy, Renilla reniformis, and Phialidium gregarium. A variety of Aequorea-related GFPs having useful excitation and emission spectra have been engineered by modifying the amino acid sequence of a naturally occurring GFP from Aequorea victoria. As noted above, other genes encoding fluorescent proteins can be used as reporter transgenes, such as, for example, yellow fluorescent protein from Vibrio fischeri strain Y-1, Peridinin-chlorophyll binding protein from the dinoflagellate Symbiodinium, phycobiliproteins from marine cyanobacteria such as Synechococcus, e.g., phycoerythrin and phycocyanin, oat phytochromes from oat reconstructed with phycoerytrobilin, β-galactosidae, alkaline phosphatase, or β-lactamase.

Nucleic acids encoding fluorescent proteins can also be obtained by methods known in the art. For example, a nucleic acid encoding a fluorescent protein can be isolated by polymerase chain reaction of cDNA from A. victoria using primers based on the DNA sequence of A. victoria green fluorescent protein. PCR methods are well known and described in, for example, U.S. Pat. No. 4,683,195 and Sambrook et al, “Molecular Cloning: A Laboratory Manual” (2001) Cold Spring Harbor Laboratory Press; 3rd edition.

Expression of reporter transgenes can be detected by a variety of methods well known to those skilled in the art. For example, fluorescence in a sample can be measured using a fluorimeter or any machine capable of measuring fluorescence. In one embodiment, detection is by fluorescently activated cell sorting (FACS) which allows recovery of cells expressing the reporter transgene. In general, fluorescence is detected by application of excitation radiation from an excitation source having a first wavelength which passes through excitation optics. The excitation optics cause the excitation radiation to excite the sample. In response, fluorescent proteins in the sample emit radiation which has a wavelength that is different from the excitation wavelength. Collection optics then collects the emission from the sample. Other means of measuring fluorescence can also be used with the invention. For example, fluorescence in living tissue can be detected using fluorescence microscopy or whole body fluorescence imaging.

The vectors of the present invention may also comprise various expression control sequences including promoters, enhancers, transcription terminators, start or stop codons, splicing signals, elements for maintenance of the correct reading frame of a gene to permit proper translation of the mRNA, and elements such as internal ribosome entry sites (‘Ires”), leader sequences and other untranslated sequences. Moreover, control sequences can be considered as part of a gene.

In addition to the use of reporter genes, there are also numerous neuronal tracers available and known to those skilled in the art for use in fate mapping of neuronal cells. For example, exemplary retrograde (mapping backwards towards the cell body) tracers include FluoroGold (Molecular Probes). Hydroxystilbamidine methanesulfonate H-22845, Diamidino Yellow (DY), Lucifer Yellow (LY), Cascade Blue (CB), Fast Blue (FB), Cholera Toxin, DiI (1,1′ dioctadecyl-3,3,3′,3′ tetramethyl-indocarbocyanine perchlorate), DiO, and Latex Nanospheres (Lumafluor).

In addition, there are also a number of anterograde (the direction from the neuron's cell body toward the axon terminal) tracers known to those skilled in the art. These include, for example, DiI (1,1 dioctadecyl-3.3.3′,3′ tetramethyl-indocarbocyanine perchlorate, DiO, Dextran conjugates, Pha-L, Biocytin. Depending on how some tracers are prepared or how they are administered, they may be used in either a retrograde or anterograde manner. These include DiI, DiO and dextran conjugates.

In addition, some of the hydrophilic tracers include FluorGold (FG), diamidino Yellow (DY), Lucifer Yellow (LY). Cascade Blue (CB) and Fast Blue (FB). These tracers may be dissolved in ddH₂O, generally in the 2-5% range. There are also lipophilic tracers available, which act by passively diffusing in-between the lipid membrane bi-layer of cells, yielding excellent labeling of neuronal processes. These lipophilic tracers are generally dissolved in organic solvents, such as DMSO, DMF, ethanol or methanol.

The dextran conjugates are taken up by cells and transported throughout the long-processes of neurons and are available in different molecular weights. Retrograde transport is most efficiently achieved with the 3,000 MW to 70,000 MW dextrans.

Wheat germ agglutinin and its derivatives, such as WGA-dsRed (discosoma red) or WGA-HRP (horseradish peroxidase) are also used for anterograde and retrograde tracing (Oztas, E., Neuroanatomy (2003), 2:2-5). In addition, the non-toxic C terminal of tetanus toxin (TTC) may also be used for neuronal tracing, and is usually used for retrograde tracing. Moreover, the non-toxic C terminal of tetanus toxin may further be conjugated to a fluorescent protein such as GFP for neuronal fate mapping or tracing studies (Roux, S. et al., Mol. Cell Neurosci. (2005), Sep; 30(1):79-89).

These tracers are injected stereotactically into specific regions of the brain, with the purpose of either retrogradely labeling of neurons with projections to the area of injection, or anterogradely labeling the projection fibers from the area of injection.

Recombinases

Any recombinase can be used in the present invention. Examples of suitable recombinases include Cre recombinase, tamoxifen inducible Cre recombinase, and Flpe. For example, a recombinase may be selected from the group consisting of CreER, CreER^{T}, CreER^{T2}, CrePR, FlpER, FlpPR, FlpePR, FlpeER, FlpeER^{T}, FlpeER^{TZ} and derivatives thereof. In particular embodiments, either the Cre loxP recombinase system of bacteriophage P1 (Lakso et al. (1992) PNAS 89:6232 6236; Orban et al. (1992) PNAS 89:6861 6865) or the FLP recombinase system of Saccharomyces cerevisiae (O'Gorman et al. (1991) Science 251:1351 1355; PCT publication WO 92/15694) can be used to generate in vivo site-specific genetic recombination systems. A preferred recombinase is an inducible recombinase, such as tamoxifen inducible Cre recombinase. Examples of non-CRE recombinases include, but are not limited to FLP recombinase of the 2μ plasmid of Saccharomyces cerevisiae, the recombination sites recognized by the resolvase family, and the recombination site recognized by transposase of Bacillus thruingiensis. Another example includes Flpe enhanced for mammals. See also Leslie Meyer-Leon, et al., “Purification of the FLP site-specific recombinase by affinity chromatography and re-examination of basic properties of the system”, Nucleic Acids Research, (1987), Vol. 15, No. 16: 6469-6488.

In the Cre-lox system, the recombination sites are referred to as “lox sites” and the recombinase is referred to as “Cre”. Cre is a site-specific DNA recombinase isolated from bacteriophage P1. The lox (locus of crossover in P1) is 34 bp in length. It has two 13 bp inverted repeats flanking an 8 bp non-palindromic core sequence. The core sequence determines the direction of the lox site. When lox sites are in parallel orientation (i.e., in the same direction), then Cre catalyzes a deletion of the intervening polynucleotide sequence. When lox sites are in the opposite orientation, the Cre recombinase catalyzes an inversion of the intervening polynucleotide sequence. Tamoxifen inducible Cre recombinase is a fusion of Cre recombinase to the ligand binding domain of the mouse or human Estrogen receptor (ER), resulting in a tamoxifen-dependent Cre recombinase that is activated by tamoxifen.

In another embodiment, a transgenic system such as a transgenic cell or transgenic animal is provided in which is present a Cre inducible recombinase transgene coding region under the control of a Gli1 gene promoter and a Cre dependent reporter transgene that is permanently expressed only in the case of Cre catalyzed recombination of DNA elements, such as LOX sites. Recombinations leading to transgene reporter expression occur in the cell expressing the Gli1 gene and mark the cell's progeny by expression of the reporter transgene regardless of whether the progeny continue to express the Gli1 gene that caused the initial expression of Cre. This methodology is an effective means of following the fate of a cell in the forward direction. In a preferred embodiment, transient activation of Cre recombinase by an activator such as tamoxefin enables Cre to catalyze a genetic rearrangement that results in EGFP expression from a constitutive promoter as well as in its progeny. The reporter transgene that is activated upon Cre expression can be obtained commercially, such as from The Jackson Laboratory (Bar Harbor, Me.). In one embodiment, the reporter transgene is carried on a vector such as an R26-lox-STOP-lox vector. In another embodiment, transgenic cells or transgenic organisms already harboring an integrated R26-lox-STOP-lox-EGFP (Soriano et al., (1999), Nature Genetics, 21: 70-71) sequence can be used in combination with a vector of the present invention or crossed with a non-human animal having inducible Cre under the control of a Gli1 gene. For example, mice containing the Cre-recombinase as descrcibed herein may be bred to R26R reporter mice, which express GFP under control of the ubiquitously active gene Rosa26 after a LoxP-flanked Stop sequence is excised by Cre (X. Mao, Y. Fujiwara, A. Chapdelaine, H. Yang, S. H. Orkin, Blood 97, 324 (2001)).

In one embodiment, the recombinase transgene encodes inducible Cre recombinase or other recombinases that can be activated by the effect of an “activator” or “inducer” such as tamoxefin. Accordingly, cells expressing the reporter transgene can be identified by detecting the expression of the reporter transgene. As will be described more fully by way of the Examples below, expression of the reporter transgene as detected by the method of the present invention correlates highly with detection of tissues wherein stem cells are present.

Vectors

The nucleotide sequence coding for a HH, or GLI or a functionally equivalent derivative, or GFP can be inserted into an appropriate expression vector, i.e., a vector which contains the necessary elements for the transcription and translation of the inserted protein-coding sequence. Thus, the nucleic acid encoding an HH or GLI or reporter protein is operationally associated with a promoter in an expression vector of the invention. Both cDNA and genomic sequences can be cloned and expressed under control of such regulatory sequences. An expression vector also preferably includes a replication origin.

The necessary transcriptional and translational signals can be provided on a recombinant expression vector, or they may be supplied by the native gene encoding the corresponding HH or GLI and/or its flanking regions. Any person with skill in the art of molecular biology or protein chemistry, in view of the present disclosure, would readily know how to assay for the expression of HH, or GLI, or a reporter protein under the control of a HH or GLI regulatory molecule, as described herein. Potential host-vector systems include but are not limited to mammalian cell systems infected with virus (e.g., vaccinia virus, adenovirus, etc.); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used. Expression of an SHH may be controlled by any promoter/enhancer element known in the art, e.g., a Simian Virus 40 (SV40) promoter, a cytomegalus virus promoter (CMV) promoter, or a tissue specific promoter such as the human glial fibrillary acidic protein promoter (GFAP) promoter, as long as these regulatory elements are functional in the host selected for expression. The resulting SHH or GLI protein or fragment thereof can be purified, if desired, by any methodology such as one that is well known in the art.

As will be apparent to those skilled in the art, the construction and use of the vectors of the present invention require transfection or transformation of cells at various times. Transformation of a host cell with recombinant DNA may be carried out by conventional techniques as are well known to those skilled in the art. For example, any of the methods known to one skilled in the art for the insertion of DNA fragments into a vector may be used to construct expression vectors encoding the polypeptides of the invention under control of transcriptional/translational control signals. These methods may include in vitro recombinant DNA and synthetic techniques and in vivo recombinations (See Sambrook et al. Molecular Cloning, A Laboratory Manual, Cold Spring Harbor Laboratory; Current Protocols in Molecular Biology, Eds. Ausubel, et al., Greene Publ. Assoc., Wiley-Interscience, NY). Where the host is prokaryotic, such as E. coli, competent cells which are capable of DNA uptake can be prepared from treated by known methods using for example, CaCl2 or MgCl2. Transformation can also be performed after forming a protoplast of the host cell or by electroporation.

When the host is a eukaryote, methods of transfection of DNA such as calcium phosphate co-precipitates, conventional mechanical procedures such as microinjection, electroporation, insertion of a plasmid encased in liposomes, or virus vectors may be used. Another method is to use a eukaryotic viral vector, such as simian virus 40 (SV40) adenovirus, vaccinia virus, or bovine papilloma virus, to transiently infect or transform eukaryotic cells and express a protein. Methods of stable transfer, meaning that the foreign DNA is maintained in the host for a suitable time, are known in the art. Further, methods by which DNA homologously recombine with the host chromosome are also known.

Eukaryotic systems, and preferably mammalian expression systems, allow for proper post-translational modifications of expressed mammalian proteins to occur. Eukaryotic cells which possess the cellular machinery for proper processing of the primary transcript, translation, glycosylation, phosphorylation, and if necessary, secretion of the gene product are preferably used as host cells for the expression of the transgene reporter.

The present invention further provides methods for using the vectors of the invention to engineer embryonic stem (ES) cells, cell lines and transgenic non-human animals from the ES cells.

Fate Mapping

In order to better understand how cells determine their destinies in any developmental process, it is important to have a reliable method of identifying particular cells and all of their descendants. While it is often the case that tissue-specific molecular markers are utilized for this purpose, it has become increasingly clear that very few markers are truly tissue specific. Moreover, it is also becoming more apparent that cells of different origins can alter their own expression pattern according to their current location (Joubin, K. et al. (1999), Cell, 98:559-571). For this reason, it is necessary to define methods that are suitable for following cells and their descendants, both during normal development and after placing them in new environments.

The preferred method for studying cell lineages would in theory be the direct observation of cells since this does not interfere with the developmental process in any way. Wetzel used time-lapse cinematography which allowed the construction of lineage trees even when specific cells could not be distinguished easily by their morphology or pigmentation (Wetzel, R. (1929), Roux' Arch. EntwMech. Org. 119: 188-321). This approach is obviously limited by the inability to identify a single cell and all of its descendants unambiguously at successive times. This approach is not possible, even with the use of time-lapse cinematography. The problems with this approach are even more apparent if one considers that it is not possible to observe cells if they have moved into the interior of an embryo, for example. Walter Vogt was the first to use vital dyes for staining living cells and for following the fate of cells in this manner (Vogt, W. (1924), Sitzungsher, Ges. Morph. Physiol. Munchen 35: 22-32; Vogt, W. Roux' Arch. EntwMech. Org. 120: 384-706). Vogt applied chips of agar, which contained dyes of different colors to the surface of urodele amphibian embryos. In this manner, small groups of cells would pick up the dye, thus allowing for their descendants to be identified even if at a later stage they were located deep inside the embryo.

Another approach utilized radioactively labeled compounds, such as tritiated thymidine. This approach involved obtaining small groups of cells from an embryo, labeling them with tritiated thymidine and then reintroducing them into the embryo (Rosenquist, G. C. (1966), Contrib. Embryol. Carnegie Institute Wash. 38: 71-110). One problem with vital dyes of the types used by Vogt and Rosenquist is that they are water-soluble. Therefore, it may be difficult to eliminate the possibility that the dyes could be transferred between adjacent, yet unrelated cells.

One solution, which was proposed and first utilized by Spemann (Spemann, H., (1921) Roux' Arch. EntwMech. Organ. 48: 533-570; Spemann, H. et al. Roux' Arch. EntwMech. Organ., 100: 599-638) and Waddington (Waddington, C. H. (1932), Phil. Trans. R. Soc. Lond. 221: 179-230) included the construction of inter-species chimeras by transplantation, which was later utilized by Le Douarin et al (Le Douarin (1973), Dev. Biol. 30: 217-222; Dupin, E. et al. ( 1998), Curr. Topics Dev. Biol. 36: 1-35). This method relied on species-specific features that allowed donor and host to be distinguished by either pigmentation (Spemann, H., (1921) Roux' Arch. EntwMech. Organ. 48: 533-570; Spemann, H. et al. Roux' Arch. EntwMech. Organ., 100: 599-638), by cell size (Waddington, C. H. (1932), Phil. Trans. R. Soc. Lond. 221: 179-230), and by the characteristic configuration of the Feulgen-positive heterochromatin associated with the nucleolus of quail cells (Le Douarin (1973), Dev. Biol. 30: 217-222). Subsequently, it was determined that improvements in the dye techniques could be made by introducing carbocyanine dyes (Axelrod, D. (1979), Biophys. J. 26: 557-573). These dyes are lipid-soluble and water-insoluble and thus partition to the membranes of cells.

Two of these dyes, DiI (red) and DiO (green) were used for the tracing of axon pathways (Godement, P. et al. (1987), Development, 101: 697-713; Thanos, S. et al. (1987), J. Comp. Neurol. 261: 155-164) and later for tracing cell lineages (Serbedzija, G. et al. (1989), Development, 106: 806-816; Yablonka-Reuveni, Z. Dev. Biol. 132: 230-240; Eagleson, G. W. et al. (1990), J. Neurobiol. 21: 427-440). Vital dyes, while they are easily applied to cells, present one problem in that it is difficult to apply them to just one cell. Thus, these dyes are often used in studies for tracking the fate of groups of cells.

The most direct method for tracking the fate of just one cell is to inject an inert tracer directly into the chosen cell, such as, but not limited to, the enzyme horseradish peroxidase (Weisblat, D. A. et al . (1978), Science, 202: 1295-1298; Lawson, K. A. et al. (1986), Dev. Biol. 115:325-339). In addition, fluorochromes conjugated to high-molecular-weight dextrans, which are used to prevent them from passing to adjacent cells through gap junctions, may be used, as well as lysine, in order to make them fixable by aldehydes (Gimlich, R. L. et al. (1985), Dev. Biol. 109: 509-514). These methods have been very successful for tracing the fates of individual cells in vertebrates as well as invertebrates. However, these procedures are technically challenging when the cell of interest is small. Another disadvantage of using enzymes or fluorescent compounds as tracers is that they become diluted as the cells divide. This problem is most noticeable in cells that divide rapidly or when the study needs to be extended over a prolonged period of time.

To overcome both problems, one viable approach using a retroviral vector to introduce the nucleic acid encoding a marker was devised by Cepko (Cepko, C. L. et al. (1984), Cell, 37: 1053-1062; Sanes, J. R. et al. (19896), EMBO J. 5:3133-3142). In this method, a retroviral vector is used to introduce a nucleic acid encoding a marker, including but not limited to alkaline phosphatase or β-galactosidase, into cells. The retroviruses are made replication-deficient by the removal of sequences coding for viral coat proteins in order to avoid the ‘secondary’ infection of cells not related to those initially marked. This approach has been used successfully, particularly to trace cell lineages in the nervous system (Cepko, C. L. et al. (1984), Cell, 37: 1053-1062).

However, there are also certain limitations to this procedure. For example, this procedure only lends itself to ‘retrospective’ lineage mapping, because the cell to be labeled cannot be chosen directly (infection is a random event among cells). A more serious problem with this approach is that even when the viral stock is greatly diluted, it is difficult to exclude the possibility that adjacent labeled cells are derived from independent infection events. Cepko et al developed a strategy to overcome this problem by constructing a complex library of about 80 different retroviruses (Golden, J. A. et al. (1995), Proc. Natl. Acad. Sci. USA, 92:5704-5708). The reasoning behind this approach was that because infection is random, the chances that two adjacent cells will be infected by the same type of retrovirus by injection of the library into tissue is significantly reduced. Despite the rigor of this approach over conventional retrovirus-mediated lineage studies, the main disadvantage remains that lineages can only be analyzed retrospectively. Additionally, it requires that every cell group be analyzed by the polymerase chain reaction to determine its relatedness to other neighboring labeled cell groups.

Another approach for tracing cell lineages retrospectively uses an infrequent, spontaneous DNA recombination event to activate the expression of a marker (β-galactosidase) in transgenic mice (Bonnerot, C. et al. (1993), C. R. Acad. Sci. 316: 1207-1217). However, this recombination event occurs at any time during development, and the labeled cell can therefore contribute to very many tissues, which complicates the analysis. To concentrate on a particular tissue, such as muscle (Nicolas, J. F. et al. (1996), Development, 122: 2933-2946) or the nervous system (Mathis, L. et al. (1997), Development, 124: 4089-4104), a tissue-specific promoter is added (so that the only cells detected are those located within the chosen tissue that are also descended from that in which the recombination event occurred).

Each of the methods for prospective lineage analysis or fate mapping, including the use of vital dyes, transplantation or intracellular injection of tracers, selects the cells to be followed on the basis of their position in the embryo at the time of labeling. It may sometimes be of interest to be able to determine the fate of cells that express a particular molecular marker, which might not all be in the same place at a particular time. One technique useful for this uses a monoclonal antibody coupled to colloidal gold in order to identify the descendants of cells that express a common surface antigen, regardless of their position in the embryo. In this procedure, antigen-expressing cells internalize the antibody-gold complex and pass it on to their descendants, thus allowing for tracing of the marker for at least a few cell divisions (Stern, C. D. et al. (1990), Nature, 343: 273-275). More sophisticated methods have since been introduced to achieve the same in ‘genetic’ organisms such as the fly, nematode and mouse. In the mouse, and as described herein, two transgenic lines are generated, one which carries a ubiquitously expressed reporter gene disrupted by a sequence delimited by LoxP sites, and another line expressing Cre recombinase under the control of a promoter that controls the expression of a gene of interest providing spatial control of marking and avoiding retrospective analysis (Zinyk, D. L. et al. (1998), Curr. Biol. 8: 665-668). When the two lines are crossed, the interrupted sequence between the LoxP sites is excised, thus activating the marker gene only in those cells in which Cre is active. This corresponds to the population of cells in which the chosen promoter is active, which may correspond to a particular cell type structure or position in the embryo. An additional modification is provided by using inducible Cre (CreER) to control the timing of marking. In this manner, one may identify and follow the fate of cells expressing the promoter at a particular time, avoiding cumulative marking.

One additional promising approach that is now being applied to many species uses the electroporation of DNA constructs into small groups of cells in vivo (Muramatsu, T. et al. (1997), Biochem. Biophys. Res. Commun. 230: 376-380). Other approaches include the use of nuclear magnetic resonance imaging to follow cells deep inside opaque embryos (Jacobs, R. E. et al. (1994), Science, 263:681-684) and the use of spectral analysis to discriminate between multiple dyes by two photon microscopy (Lansford, R. et al. (2001), J. Biomed. Optics, 6: 311-318) (see http://bioimaging.caltech.edu/microscopy.html#spectroscopy).

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### Production of Transgenic Mice

Gli1-CreER^{T2} Knockin Mice

Gli1-CreER^{T2} mice were generated according to Ahn and Joyner (Ahn, S and Joyner, A. L. (2004), Cell 118: 505-516). Briefly, a 2.3 kb EcoR1-Hsp921 5′ Gli1 genomic fragment immediately upstream of the ATG translation start site was subcloned into the pBKS vector to make pKS-Gli1 5′. A cDNA encoding CreER^{T2} (Feil, R., Wagner, J., Metzger, D., and Chambon, P. (1997). Regulation of Cre recombinase activity by mutated estrogen receptor ligand-binding domains. Biochem Biophys Res Commun 237, 752-757) was fused to a triple SV40 poly A240 bp fragment using the Sacl site in the vector. This 2.8 kb CreER^{T2}-pA3 fragment was then subcloned into pKS-Gli1 5′ using the Sal1/Kpn1 sites. A 4.5 kb EcoR1 Gli1 3′ genomic fragment containing exons 1-6 was subcloned into pPNT-loxP-Neo-loxP using the EcoR1 site to create pPNTflox-neo-3′. The 5.1 kb fragment of Gli1 5′-CreER^{T2}-pA3 was then subcloned into pPNTflox-neo-3′ suing the Sal1/Noit1 sites to create the final targeting vector. The neo cassette is in the opposite transcriptional orientation to the endogenous Gli1 gene. Homologous recombination in W4 ES cells was performed as described previously (Matise, M. P., Auerbach, W., and Joyner, A. L. (2001). Production of targeted embryonic stem cell clones. In Gene Targeting, A. L. Joyner, ed. (Oxford Univerisity Press), pp. 101-132). Seven targeted ES clones (Gli1-CreER^{T2}-neo) were identified from 121 G418 and gancyclovir resistant clones by Southern blot analysis using both 5′ and 3′ external probes on Xhol- and Xbal-digested genomic DNA, respectively. Three ES cell clones were injected into C57BL/6 blastocysts to generate ES cell chimeras (Matise, M. P., Auerbach, W., and Joyner, A. L. (2001). Production of targeted embryonic stem cell clones. In Gene Targeting, A. L. Joyner, ed. (Oxford Univerisity Press), pp. 101-132), and successful germ line transmission was accomplished from all three independent ES cell clones. The neo cassette was removed by crossing Gli1-Cre^{T2}-neo heterozygotes with TK-Cre Black Swiss mice as described (Bai, C. B., Auerbach, W., Lee, J. S., Stephen, D., and Joyner, A. L. (2002). Gli2, but not Gli1, is required for initial Shh signaling and ectopic activation of the Shh pathway. Development 129, 4753-4761). Routine genotyping was performed using PCR for Cre as described (Kimmel, R. A., Turnbull, D. H., Blanquet, V., Wurst, W., Loomis, C. A., and Joyner, A. L. (2000). Two lineage boundaries coordinate vertebrate apical ectodermal ridge formation. Genes Dev 14, 1377-1389). FIG. 9 shows a schematic drawing of the transcripts and products of Gli1.

R26R Mice

R26R mice were generated according to Soriano (Soriano, P. (1999), Nat. Genet 21:70-71).

Animals generated were handled and housed according to institutional guidelines.

### Example 2

### Fate Mapping Studies

Two-three month old Gli1-CreERT2/+; R26R/R26R adult mice were given 10 mg of tamoxifen (TM; Sigma) by oral gavage in corn oil on two consecutive days using feeding needles (FST). For AraC treatment, two days after TM treatment, a mini-osmotic pump (model 1007D, Duret) was implanted to infuse either saline or 2% AraC (Sigma) for one week as described (Doetsch, F. et al. (1999), Proc Natl Acad Sci USA 96: 11619-11624). Brains of mice that were sacrificed at the end of AraC or control treatment were designated as day 0 samples. For long-term survival experiments, the pump was removed one week after implantation and mice were later sacrificed at the indicated survival time. The second application of AraC was done as the first AraC treatment.

### Example 3

### Immunostaining and Analysis of Brain Tissues

Mice were perfused (4% paraformaldehyde (PFA)), processed for frozen sections, and stained with X-gal as described in the Joyner lab website (http://saturn.med.nyu.edu/research/dg/joynerlab). For immunofluorescent staining, dissected brains were fixed in 4% PFA overnight at 4° C. and 50 μm vibratome (Leica) sections were obtained. Free-floating sections were stained overnight at 4° C. with goat anti-galactosidase (1:500; Biogenesis) and one of the following antibodies: mouse anti-GFAP (1:500), anti-PSA-NCAM (1:300), anti-NeuN (1:200; Chemicon), anti-CCI (1:20, Calbiochem), anti-CNPase (1:150), anti-MBP (1:200, SMI), guinea pig anti-Olig2 (1:20,000; from B. Novitch), rat anti-NG2/AN2 (1:10; from C. Klein), and rabbit anti-GABA (1:500, Sigma). Secondary antibodies for double labeling were donkey anti-species conjugated with Alexa 488, Alexa 555 (1:500; Molecular Probes), or FITC (1:200; Jackson ImmunoResearch). Fluorescent images were captured in 1.5-μm optical sections using a confocal laser-scanning microscope (LSM 510, Zeiss) and processed with Adobe Photoshop. Orthogonal analysis was performed to confirm co-expression of two markers. Cells co-expressing lacZ and one of the various markers were counted and divided by total number of lacZ expressing cells in the indicated region. At least three sections through the SVZ and DG were analyzed from one mouse. Data from at least three mice were pooled together to determine the average and standard deviation (SD). T-test was performed to calculate the p-value and determine whether the results between two time points were different with statistical significance.

Results and Summary

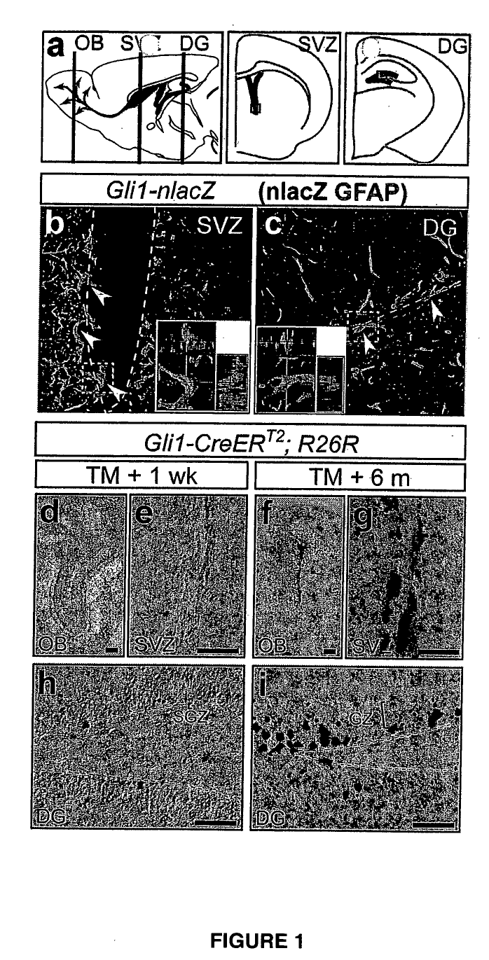

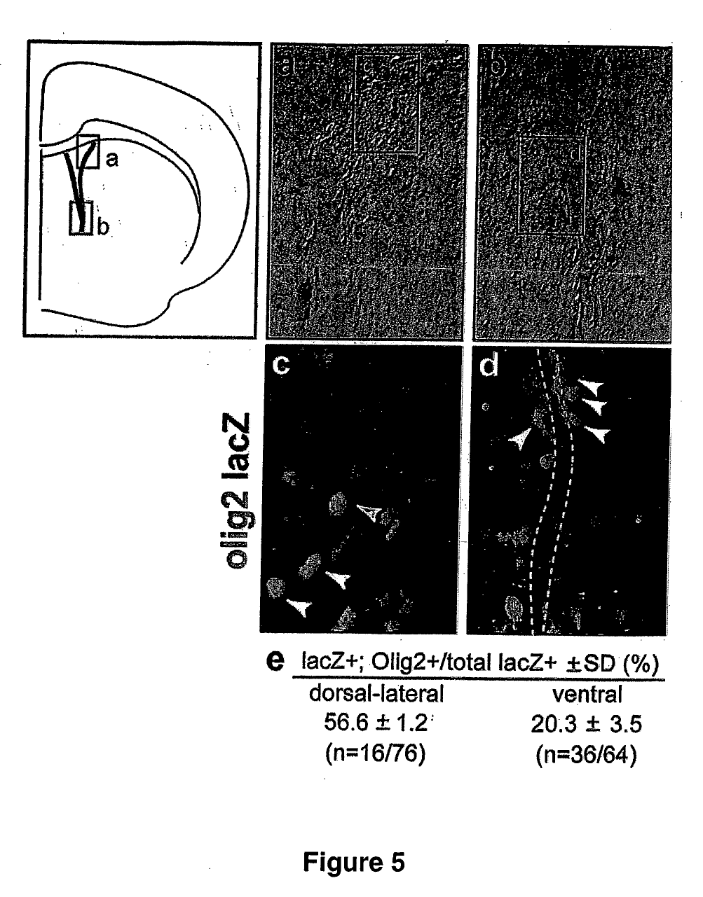

In order to investigate whether NSCs normally respond to Shh in the adult mouse brain, genetic techniques to identify cells responding to Shh were utilized. First, the SVZ and SGZ of adult mice (n=3) were analyzed for co-expression of Gli1-nlacZ, a sensitive read-out of positive Shh signaling (Bai, C. B., Auerbach, W., Lee, J. S., Stephen, D. & Joyner, A. L., Development 129, 4753-61 (2002); Bai, C. B., Stephen, D. & Joyner, A. L., Dev Cell 6, 103-15 (2004) and GFAP that marks NSCs (Doetsch, F., Caille, I., Lim, D. A., Garcia-Verdugo, J. M. & Alvarez-Buylla, A., Cell 97, 703-16 (1999); Seri, B., Garcia-Verdugo, J. M., McEwen, B. S. & Alvarez-Buylla, A., J Neurosci 21, 7153-60 (2001), in addition to mature astrocytes. Shh-responding (nuclear lacZ+) cells in the ventral SVZ and SGZ co-expressed GFAP (25.3% and 18.8%, respectively), suggesting that NSCs respond to Shh activity (FIG. 1b and 1c). In addition, 18% and 36% of Shh-responding cells in the SVZ and SGZ co-expressed PSA-NCAM (Table 1), a neuroblast/neural precursor marker. Co-labeling with Dlx2 (Doetsch, F., Petreanu, L., Caille, I., Garcia-Verdugo, J. M. & Alvarez-Buylla, A., Neuron 36, 1021-34 (2002)), (data not shown), or Olig2 (Hack, M. A., Sugimori, M., Lundberg, C., Nakafuku, M. & Gotz, M., Mol Cell Neurosci 25, 664-78 (2004)) (FIG. 5; 57% of Shh-responding cells), transit-amplifying cell markers, showed that fast-dividing transit-amplifying cells also respond to Shh signaling. However, there were no Shh-responding cells found in the RMS and OB, the target structures of the cells generated in the SVZ, suggesting that NSCs and their immediate precursor cells respond to Shh activity.

The only definitive test that Shh signals to NSCs in vivo would be to mark the Shh-responding cells and determine whether they self-renew and continuously give rise to mature neurons. To mark and follow the Shh-responding cells in the SVZ and SGZ in vivo, Gli1-CreER^{T2} mice, in which an inducible Cre recombinase (CreER^{T2}) is expressed from Gli1, were used for genetic fate mapping (Ahn, S. & Joyner, A. L., Cell 118, 505-16 (2004)). Upon tamoxifen (TM) treatment of Gli1-CreER^{T2}; R26R mice, CreER^{T2} is transiently activated for ˜30 hrs resulting in permanent expression of cytoplasmic lacZ from the R26R locus in Gli1-expressing cells and all their progeny (Ahn, S. & Joyner, A. L., Cell 118, 505-16 (2004); Zervas, M., Millet, S., Ahn, S. & Joyner, A. L., Neuron 43, 345-57 (2004)). Initially, a small number of Shh-responding cells in the SVZ and SGZ of adult Gli1-CreER^{T2}; R26R mice (n=3) were labeled (FIGS. 1e and 1h). Due to the mosaic nature of our genetic fate mapping approach, not all Shh-responding cells are labeled with TM (Ahn, S. & Joyner, A. L., Cell 118, 505-16 (2004); Zervas, M., Millet, S., Ahn, S. & Joyner, A. L., Neuron 43, 345-57 (2004)). However, marker expression analysis of the initial population revealed that the distribution of different marked cell types was similar to that of Gli1-nlacZ mice (Table 1). Thus, the initially marked population faithfully recapitulates the true representation of Shh-responding cells in the SVZ and SGZ.

The long-term fate of the marked cells analyzed six months after TM treatment demonstrated a dramatic increase in the number of labeled cells in Gli1-CreER^{T2}; R26R mice (n=5) in both the SVZ and DG (FIGS. 1g, 1i, and data not shown). In addition, while no labeled cells were detected in the RMS or OB at the initial time point (FIG. 1d and data not shown), long-term fate mapping showed labeled cells throughout the RMS and OB (FIG. 1f and data not shown), demonstrating that the small initial population of Shh-responding cells in the SVZ amplified greatly to generate labeled cells in the OB that had migrated via the RMS. Also, in the DG of the hippocampus, many labeled cells integrated into deeper layers of the granular zone after six months (FIG. 1h, black bracket) compared to the initial Shh-responding population primarily confined to the SGZ (FIG. 1i, red bracket). Taken together, our long-term fate mapping results indicate that Shh-responding cells in the SVZ and SGZ include NSCs that can generate progeny over time in their respective target structures.

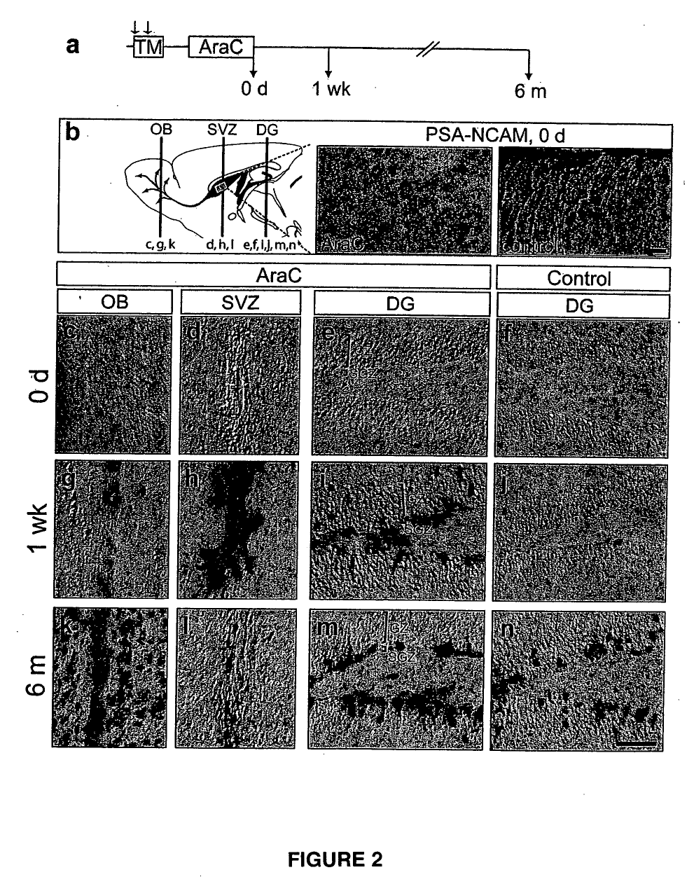

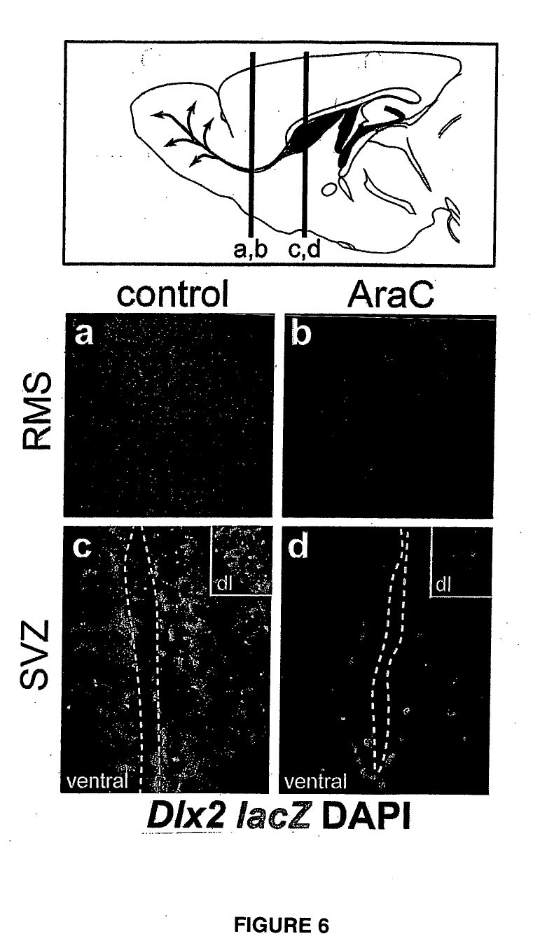

To determine whether Shh-responding cells include the quiescent NSCs that rarely divide, rather than only the fast-dividing transit-amplifying cells with a limited life span, we utilized AraC, an antimitotic reagent that effectively kills fast-dividing cells in the SVZ and SGZ while sparing quiescent NSCs (Doetsch, F., Garcia-Verdugo, J. M. & Alvarez-Buylla, A., Proc Natl Acad Sci U S A 96, 11619-24 (1999)). Two days after TM treatment, AraC was infused for one week into the brain of Gli1-CreER^{T2}; R26R mice using a mini-osmotic pump. At the end of AraC treatment (day 0), PSA-NCAM immunoreactivity in whole mounts of the lateral wall of the lateral ventricles (n=3) revealed that the neuroblasts were almost completely depleted (FIG. 2b and data not shown), confirming the efficacy of AraC treatment. Elimination of fast-dividing precursors/neuroblasts by AraC treatment was confirmed on sections through the SVZ and DG where only one of 69 and one of 63 lacZ+ cells, respectively, showed PSA-NCAM immunoreactivity (Table 1). In addition, Dlx2 expression was abolished in the SVZ and greatly diminished in the RMS (FIG. 6 and data not shown). Importantly, at the end of AraC treatment, 92.2±1.0% and 93.7±4.7% of the remaining labeled cells in the SVZ and SGZ, respectively, co-expressed GFAP (data not shown), indicating that quiescent NSCs respond to Shh signaling in vivo. Strikingly, within one week after AraC removal (n=5), labeled cells in the SVZ and DG of AraC treated mice expanded more than control mice (n=4) (FIG. 2h-j and data not shown). Compared to no labeled cells in the OB at the end of AraC treatment, labeled cells were found in the granule cell layer in the OB one week after AraC removal (FIG. 2g). The extent of labeling in AraC treated mice, while continuing to expand, returned to a comparable level as in the control mice at two months (n=3 each; data not shown), three months (n=4 each; data not shown) and six months (n=3 each; FIG. 2k-n). These results provide additional strong evidence that Shh-responding cells in the SVZ and SGZ that survived AraC treatment are rarely-dividing NSCs and respond to this insult by increasing cell proliferation and the production of transit-amplifying cells.

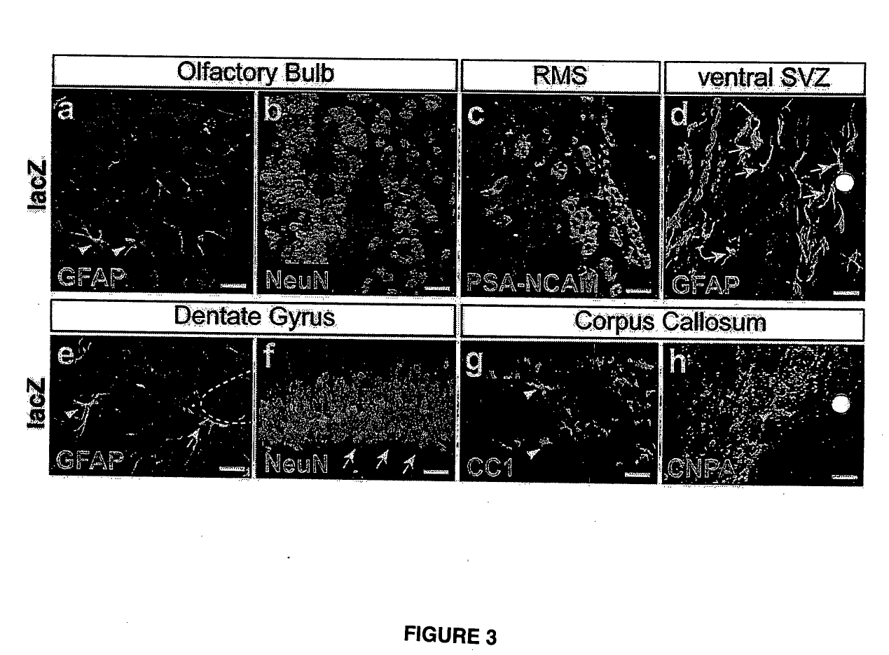

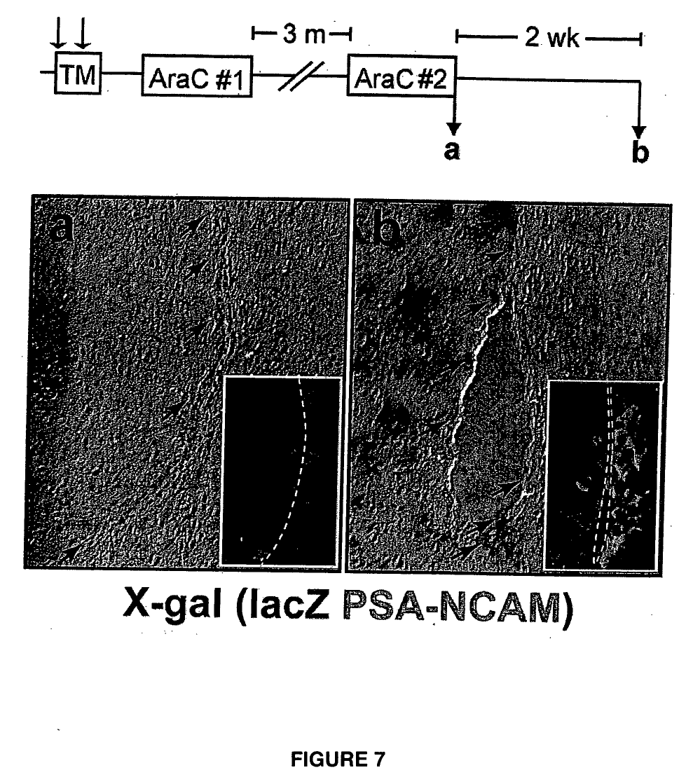

Even though NSCs isolated from the SVZ and SGZ exhibit characteristics of stem cells in vitro, including self-renewal and generation of multiple cell types (Seri, B., Garcia-Verdugo, J. M., McEwen, B. S. & Alvarez-Buylla, A., J Neurosci 21, 7153-60 (2001); Palmer, T. D., Takahashi, J. & Gage, F. H., Mol Cell Neurosci 8, 389-404 (1997); Gage, F. H., Kempermann, G., Palmer, T. D., Peterson, D. A. & Ray, J., J Neurobiol 36, 249-66 (1998)), it has not been demonstrated whether quiescent NSCs in vivo self-renew or generate cell types other than neurons in their respective target structures (Suhonen, J. O., Peterson, D. A., Ray, J. & Gage, F. H., Nature 383, 624-7 (1996); Herrera, D. G., Garcia-Verdugo, J. M. & Alvarez-Buylla, A., Ann Neurol 46, 867-77 (1999)). Previous lineage tracing experiments have relied on rare infection of NSCs and transit-amplifying cells with replication defective retroviruses (Yoon, S. O. et al., Proc Natl Acad Sci U S A 93, 11974-9 (1996); Doetsch, F., Garcia-Verdugo, J. M. & Alvarez-Buylla, A., J Neurosci 17, 5046-61 (1997)), or BrdU labeling of progenitor cells in their last division (Kuhn, H. G., Dickinson-Anson, H. & Gage, F. H., J Neurosci 16, 2027-33 (1996); Kempermann, G., Gast, D., Kronenberg, G., Yamaguchi, M. & Gage, F. H., Development 130, 391-9 (2003)) to determine the long term fate of NSCs. We took advantage of our highly efficient method for marking quiescent NSCs in the adult to determine their long-term fate after one year (FIG. 3). We first concentrated on the self-renewal capacity of the quiescent NSCs that respond to Shh signaling. The percentage of labeled cells that co-express GFAP increased significantly (p<0.05) one year after marking in the SVZ (from 23.2±1.6% initially to 34.2±4.7% at one year) and in the SGZ (18.8±3.3% initially to 30.3±5.3% at one year). In addition, the absolute number of labeled cells co-expressing GFAP in the SVZ and SGZ increased, suggesting that Shh-responding NSCs self-renewed to generate more NSCs in both AraC-treated and control mice (FIGS. 3d, 3e and Table 1). To further demonstrate the ability of Shh-responding NSCs to self-renew, Gli1-CreER^{T2}; R26R mice (n=2) that were treated with AraC following TM administration were allowed to recover for three months and subjected to another round of AraC treatment. Even after the second round of elimination of the fast-dividing precursors, the remaining NSCs that were marked prior to the first AraC treatment survived and generated more PSA-NCAM+ neuroblasts (FIG. 7). Taken together, these data demonstrate that quiescent NSCs respond to Shh signaling and self-renew in vivo.

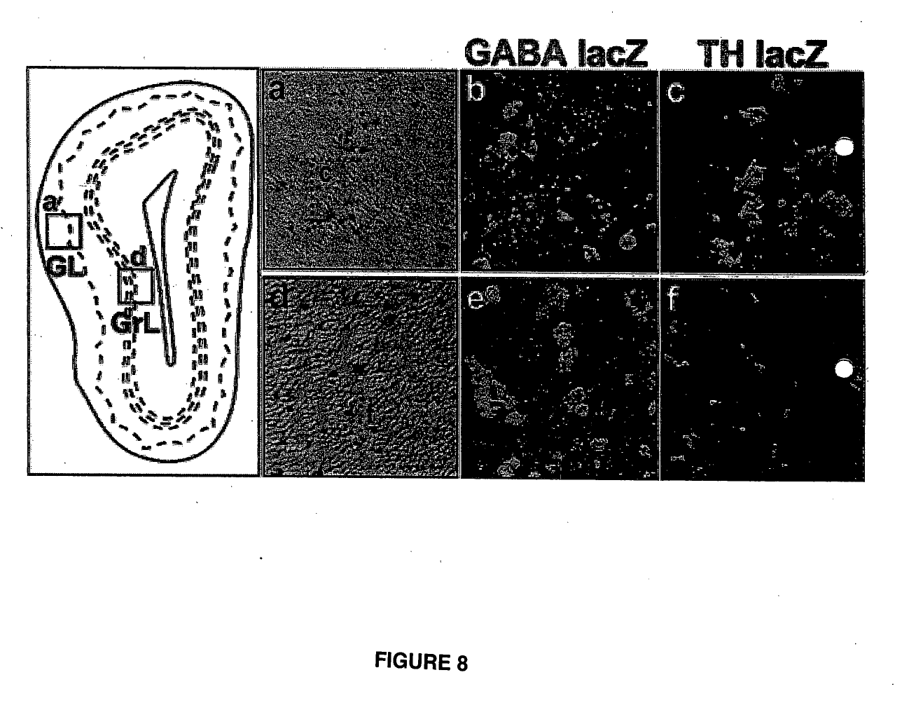

In addition to the GFAP+ cells that remain in the SVZ, many PSA-NCAM and lacZ co-expressing cells were observed in the dorsal-lateral SVZ (data not shown), where neuroblasts exit the SVZ to enter the RMS en route to the OB. In the anterior RMS, most of the labeled cells were co-labeled with PSA-NCAM (FIG. 3c). Thus, quiescent NSCs in the SVZ actively produce new neuroblasts even one year after marking. In the OB, the vast majority of labeled cells were neurons as evidenced by their co-expression of lacZ and NeuN (92.3±4.5%; FIG. 3b), as well as their elaborate dendritic arborizations. In the periglomerular region, there was extensive co-expression of lacZ and GABA or TH, whereas labeled granular neurons mostly expressed only GABA (FIG. 8). We also detected a few GFAP-expressing fate-mapped cells within the OB (3.5±0.5%; n=3) (FIG. 3a). This suggests that while most of the progeny generated from the SVZ become interneurons of the OB, some also become astrocytes in the OB. In addition, there were a few marked oligodendrocytes in the corpus callosum (FIGS. 3g and 3h). In the DG, the vast majority of fate-mapped cells co-expressed NeuN (82.2±7.8%), indicating that they became granule neurons in the deeper layers of the granular zone (FIG. 3f). Surprisingly, a few lacZ-expressing cells in the deeper layer of the granular zone also expressed GFAP (3.3±0.7%), suggesting the NSCs also generated astrocytes in vivo (FIG. 3e and Table 1). Our results demonstrate for the first time in vivo that quiescent NSCs have the potential to generate multiple cell types.

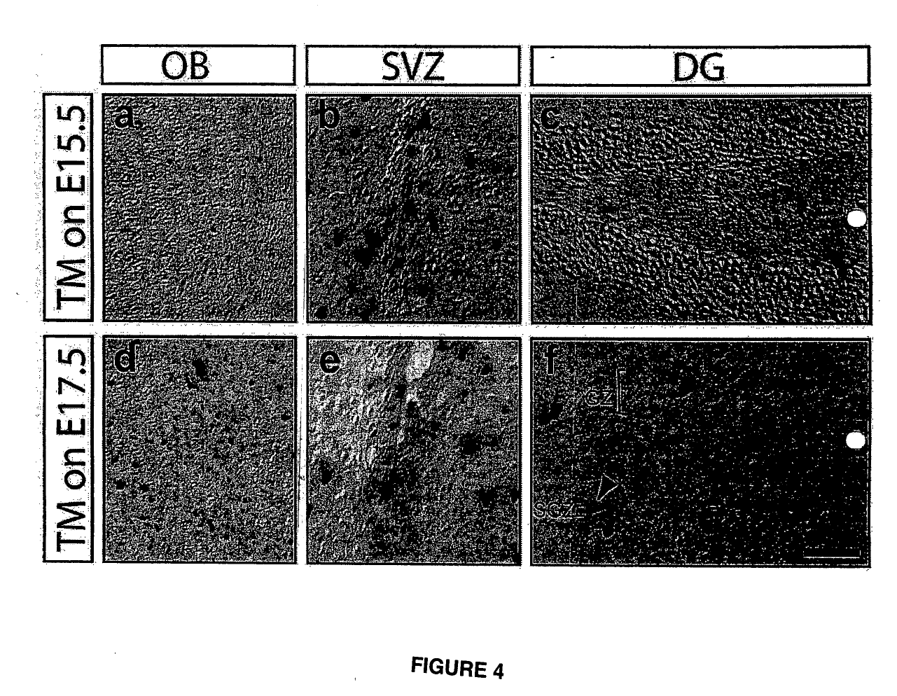

Our genetic fate mapping technique provides the first opportunity to explore the genetic history of NSCs and formation of neural stem cell niches. To determine whether adult NSCs in the SVZ and DG are derived from cells that had previously responded to Shh in the embryo, we analyzed the long-term fate of Shh-responding cells marked at various embryonic stages. No labeled cells were found in the adult SVZ and SGZ when TM was given between E7.5 and E11.5 (data not shown). The appearance of marked cells was first observed in the SVZ when TM was given at E15.5, whereas labeled cells in the SGZ did not appear until TM was given at E17.5 (FIG. 4). This demonstrates that Shh does not act on forebrain NSCs until late embryogenesis and further suggests that the two stem cell niches in the forebrain are established sequentially in mice. In addition, we found that the long-term fate of the Shh-responding cells in the DG marked at E17.5 exhibited a different behavior from cells marked in the adult: The cells marked with TM on E17.5 populated deeper layers of the granular zone of the DG than cells marked in the adult (compare FIGS. 1i, 2n and 4f, black brackets) suggesting a temporal restriction in the extent of integration of newly-generated cells as the brain matures.

Our genetic fate mapping studies demonstrate that Shh first acts on stem cell niches in the SVZ and SGZ at late embryonic stages. Quiescent neural stem cells are then set aside and regulated by Shh signaling. Importantly, we present the first in vivo evidence that the NSCs can self-renew for at least a year and generate multiple cell types over time. Hh signaling has recently been implicated in the stem cell biology associated with tissue repair and progression of tumors in many non-neural tissues (Berman, D. M. et al., Nature 425, 846-51 (2003); Karhadkar, S. S. et al., Nature 431, 707-12 (2004); Watkins, D. N. et al., Nature 422, 313-7 (2003)). Our in vivo genetic fate mapping approach provides a unique opportunity to determine whether in fact stem cells regulated by Hh signaling play a key role in cell replenishment of various organs, and to elucidate the mechanism by which they contribute to tissue repair and cancer.

Summary

An in vivo genetic fate mapping strategy using Gli1 as a sensitive read-out of Shh activity was utilized to systematically mark and follow the fate of Shh-responding cells in the adult mouse forebrain. It has been demonstrated here that only a small initial population of cells that include both quiescent neural stem cells (NSCs) and transit-amplifying cells responds to Shh in regions undergoing neurogenesis, but subsequently expands dramatically to continuously provide new neurons in the forebrain. The studies of the behavior of quiescent NSCs provides the first in vivo evidence that they can self-renew for over a year and generate multiple cell types. Furthermore, these studies show that the NSC niches in the subventricular zone and dentate gyrus are established sequentially and not until late embryonic stages. Exemplary GenBank accession numbers for sequences that may be employed for use in embodiments of the invention are found in Table 2.

TABLE 1

| | | Quantification of the fate-mapped cells | | | | |
|---|---|---|---|---|---|---|
| | | SVZ*DG | | | | |
| | | | | | lacZ+;GFAP +/ | |
| | Actual # | lacZ+;GFAP + | | | total | |
| | analyzed | / total lacZ+ ± | # of | Actual # | lacZ+ ±SD | # of |
| a. NSCs | § | +SD (%) | mice | analyzed § | (%) | mi |

| | | | | | | |
|---|---|---|---|---|---|---|
| Gli1-nlacZ | 71/284 | 25.2 ± 5.3 | 3 | 15/71 | 18.8 ± 3.3 | 3 |
| Control 0 | 15/63 | 23.2 ± 1.6 | 3 | 24/106 | 23.0 ± 3.8 | 3 |
| day | | | | | | |
| AraC 0 day | 54/58 | 92.2 ± 1.0 | 3 | 42/45 | 93.7 ± 4.7 | 3 |
| Long-term | 74/217 | 34.4 ± 4.7 | 4 | 47/245 (DG) | 19.4 ± 7.9 | 55 |
| | | | | 34/111 | 30.3 ± 5.3 | |
| | | | | (SGZ*) | | |

| | | lacZ+; PSA- | | | lacZ+; PSA- | |
|---|---|---|---|---|---|---|
| b. | Actual # | NCAM+/ | | | NCAM+/ | |
| neuroblast/ | analyzed | total lacZ+ ± | # of | Actual # | total lacZ+ ± | # of |
| precursor | § | SD (%) | mice | analyzed § | SD (%) | mi |
| Control 0 | 8/44 | 18.2 | 2 | 18/47 | 38.2 ± | 3 |
| day | | | | | 1.8 | |
| AraC 0 day | 1/69 | 1.6 ± 2.9 | 4 | 1/63 | 2.1 ± 3.6 | 4 |
| Long-term | 22/120 | 18.5 ± 7.3 | 4 | 37/116 | 32.4 ± | 3 |
| | | | | | 5.0 | |

| | | | | |
|---|---|---|---|---|
| | | lacZ+; GFAP+/ total | | lacZ+; PSA- |
| | Long- | lacZ+ ± SD (%) (Actual # | lacZ+; NeuN+/total | NCAM+/total |
| c. | term | analyzed§; # mice) | lacZ + ± SD | lacZ+ ± SD |

| | | | | | | |
|---|---|---|---|---|---|---|
| Olfactory | 3.5 ± 0.5 (5/140; 3) | 92.3 ± | (271/ | 3) | ND 32.4 ± 5.0% | 3 |
| | | | 288; | | | |
| bulb | SGZ**: 30.3 ± 5.3 | 4.5% | 88; | | (37/116; | |
| Dentate | (34/111;5) GZ***: 3.3 ± | 82.2 ± | (142/ | 4) | | |
| | | | 174; | | | |
| gyrus | 0.6 (8/234; 5) | 7.8% | 74; | | | |

§ Total number of lacZ+; marker+ cells/ total number of lacZ+ cells
*SVZ (subventricular zone) quantification is done in the ventral SVZ.
**SGZ (subgranular zone) is defined as the inner layer of the DG with 1-2 cell width.
***GZ (granular zone) is defined as the high-density granule cell layer of the dentate gyrus

### Example 4

### Stromal Cells of the Developing and Regenerating Prostate Respond to Shh and Require Gli1

Studies were done to determine whether Shh has a general function in adult stem cells. It is thought that most adult organs have dormant stem cells, but the nature of these stem cells and their normal function in response to injury or involvement in cancer are not well understood. We have concentrated on epithelial organs, many of which have ducts lined with epithelial cells and are surrounded by mesenchymal cells (stroma). In many such tissues (e.g. lung, gut, prostate, kidney) the stroma consists of two cell types: smooth muscle cells that wrap the ducts and loose fibroblasts in the intersticial space. In many epithelial tissues Shh is expressed in the epithelial cells, and Gli1 in the stroma. While the existence of epithelial stem cells has been demonstrated in structures such as the gut where there is a high cell turn over rate of the epithelium, in most tissues there appears to be little turn over of the epithelium and thus the existence of dormant epithelial stem cells has not been extensively documented. Little also is known about whether stromal stem cells exists in soft tissues, and whether such a cell can give rise to both smooth muscle and fibroblasts. In the bone marrow, numerous studies have shown that stromal stem cells can give rise to a variety of mesenchymal tissues and cell types (including fibroblasts), but whether they can give rise to non-mesenchymal cells in vivo remains controversial (Gimble, J M et al. (2006), J. Cell Biochem., May 15: 98(2):251-266).

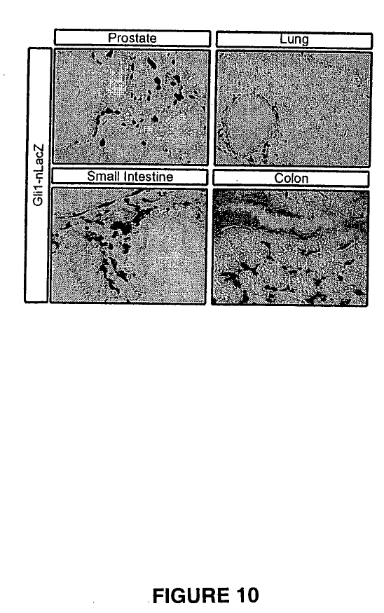

As a first approach to determine whether Shh signals to stem cells in diverse adult tissues, we analyzed lacZ expression on sections of tissues from Gli1-lacZ mice and found lacZ positive cells scattered throughout the stroma. Based on the shape and position of the lacZ-expressing cells, Shh appears to signal to both smooth muscle and fibroblasts (FIG. 10). We then used Genetic Inducible Fate Mapping (GIFM) as described above using Gli1-CreER;R26 mice to mark adult Gli1-expressing cells and found a small number of LacZ positive cells in the stroma of a range of organs including prostate and lung at the initial time period (1 week after two injections of 10 mg of tamoxifen (TM)) and very little (2-3 fold), if any, increase in the number of marked cells after a year (data not shown). While this result suggests that during homeostasis stromal cells expand little, it does not preclude that they proliferate in response to injury, or that Gli1 is expressed in rare dormant epithelial stem cells.

### Example 5

### Fate Mapping Shh Responding Cells During Postnatal Prostate Development

To further explore the existence of stromal stem cells in adult epithelial tissues, we chose to concentrate on the prostate because of the high incidence of prostate cancer and BPH, and because Shh is required for expansion of embryonic stroma. In addition, the prostate offers a distinct advantage for studying dormant adult stem cells, in that it can be induced to undergo involution and regeneration by castration and subsequent administration of Androgens. Furthermore, involution/regeneration can be induced at least 20 times by withdrawing and administering Androgens.

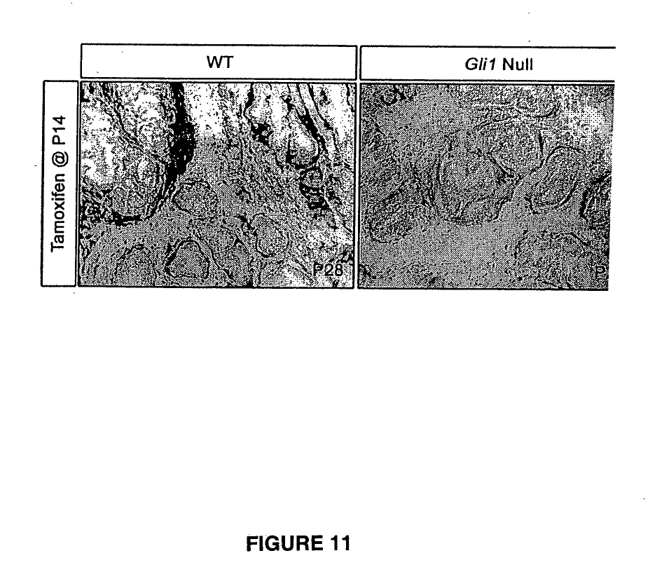

Gli1^{CreER/+};R26R mice were administered TM on postnatal day (P) 14 and prostate sections stained with X-gal at P28 to determine the contribution of the Shh-responding cells to the developing prostate. Our data indicate that while at the initial time point (2 days post TM) only a small number of lacZ+ cells were observed, at P28 there was a significant contribution of the Gli1 derived cells to the glandular stroma (FIG. 11). Based on the morphology of the marked cells, they appear to include both smooth muscle and fibroblasts. This result indicates that during postnatal expansion of the prostate, Shh-signaling regulates expansion of the stroma.

### Example 6

### Shh-Responding Stromal, and Not Epithelial, Cells Expand During Prostate Regeneration

As an initial approach to determine whether a dormant stromal stem cell population exists in the adult prostate, we used GIFM to follow the fate of Shh-responding stromal cells during regeneration following involution caused by castration. This approach also allowed us to test whether rare epithelial stem cells are marked by Gli1-CreER, since epithelial stem cells proliferate during regeneration. Such rare cells would therefore expand and produce extensive lacZ+ marking of the prostate epithelium.

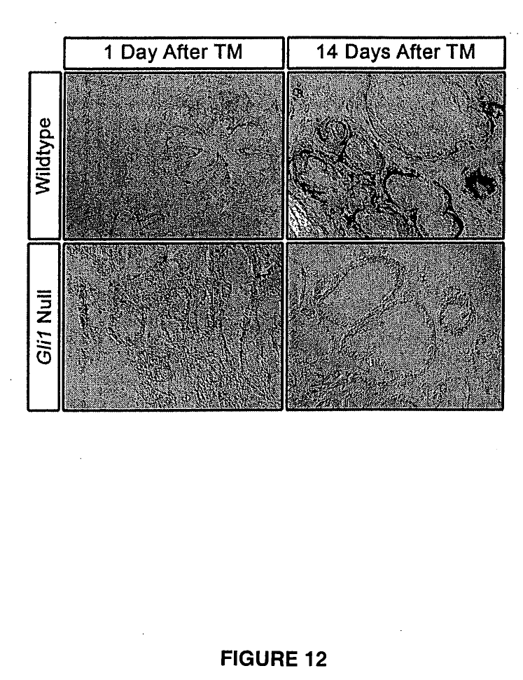

Two month old Gli1^{CreER/+};R26R animals were castrated, and after a two-week involution period, subcutaneous androgen pellets were implanted and TM administered for three consecutive days to mark Shh-responding cells. At the initial time point (short term marking 1 day after TM) there were few stromal cells labeled (FIG. 12—arrows point to lacZ+ cells). In contrast, after regeneration (long term marking after 14 days) there was a significant expansion of the labeled cells. Furthermore, the lacZ+ cells appeared to be confined to the stroma and excluded from the epithelium. The lack of marking in the epithelial compartment is consistent with the proposal that Smo is not expressed in the epithelial layer, and further indicates that rare epithelial stem cells do not normally respond to Shh. Our preliminary double labeling experiments show that Beta-galactosidase co-localizes with a marker of smooth muscle cells (SMA) in many cells, but never with a marker of basal epithelial cells (CK5) (data not shown). This indicates that in the adult prostate only the stromal cells (smooth muscle and fibroblasts) respond to Shh.

### Example 7

### Gli1 is Required for Normal Prostate Postnatal Stromal Development

As a first approach to determine whether Shh-signaling is required for the expansion of stromal cells during postnatal prostate development and regeneration following involution, we asked whether Gli1 is required for these processes. Strikingly, we found that in Gli1^{CreER/CreER} mice (Gli1 null mutants) carrying R26R and treated with TM at P14 and analyzed at P28 the contribution of Gli1-CreER marked cells (lacZ+) appeared to be significantly reduced compared to Gli1^{CreER/+};R26R animals. Furthermore, the overall stromal compartment in these animals appeared reduced, with large patches of tissue containing almost no stroma and the epithelium appeared abnormal. The latter likely reflects a reduction in critical signaling from the stroma to the epithelial cells. As expected, Gli1^{nLacZ/nLacZ} null mutant mice exhibited the same stromal depletion phenotype found in the Gli1^{CreER/CreER} null mutants. Furthermore, when 2 month old Gli1^{CreER/CreER};R26R animals were castrated and androgens and TM administered, whereas a similar number of lacZ positive stromal cells were seen in the short term labeling as in wild types, no expansion of these cells was observed after 2 weeks, and instead there appeared to be a reduction in labeled cells. The epithelial component of the glands in these mice appeared smaller and irregularly shaped, and the individual epithelial cells appeared smaller, consistent with a functional reduction in their secretory activity. Thus, the reduced stromal phenotype seen in our developmental studies was exaggerated after regeneration.

### Example 8

### Fate Mapping of Hedgehog Responding Cells Using Gli1-EGFP Transgenic Mice

We have used a second mouse model for monitoring hedgehog responding cells. These mice express GFP in cells responding to Shh. This allows us to sort specifically for cells responding to Shh based on GFP expression. They can be used in microarray studies to identify genes induced by Shh, or specific to a particular cell type. They can also be used to determine whether they are stem cells. These mice were made using the procedure as outlined above for the Gli1-CreER mice, except that GFP replaced the CreER and no Gli1 intron sequences were removed.

TABLE 2
GENBANK REFERENCE NUMBERS

| | |
|---|---|
| Gli 1 Mouse: | NM_010296 |
| Gli 1 Mouse: | NP_034426 |
| Gli 1 Rat: | BC_089858 |
| Gli 1 Rat: | XM_233779 |
| Shh Mouse: | NM_009170 |
| Shh Rat: | NM_204821 |
| Smoothened: | NM_176996 |
| Green Flourescent Protein: | CAA58790, L29345 |
| Green Flourescent Protein: | L29345 |
| Green Flourescent Protein: | M62654 |
| Enhanced GFP: | BAB11884 |
| Red Fourescent protein: | AF272711 |
| Red Fourescent protein: | AAG16224 |
| Red Fourescent protein: | AY015995 |
| Beta galactosidase: | NM_009752 |
| Luciferase: | E08320 |
| Luciferase: | E08319 |
| Yellow Flourescent protein: | M60852 |
| Yellow Flourescent protein: | E08319 |
| Wheat Germ agglutinin | E05282 |
| HRP | E01651 |
| Tetanus toxin | AF154828 |
| Alkaline Phosphatase | NM_007433 |
| Alkaline Phosphatase | BC_065175 |
| Alkaline Phosphatase | BC_119800 |
| Alkaline Phosphatase | BC_088399 |

What is claimed is:

1. A transgenic non-human animal whose genome comprises a transgene comprising a nucleic acid sequence encoding the Gli1 gene transcriptional regulatory sequences, an inducible site specific recombinase operably linked to a Gli1 promoter enhancer, and a reporter gene operably linked to a promoter that directs expression of the reporter gene in a cell of the transgenic animal and its progeny during development and in the adult animal, wherein the reporter is only expressed in a cell that has a functional Cre recombinase.

2. The transgenic non-human animal of claim 1, wherein the Gli1 promoter enhancer directs the expression of a site-specific recombinase in at least one cell of the transgenic non-human mammal responding to the family of Hedgehog proteins.

3. The transgenic non-human animal of claim 1, wherein the Gli1 promoter enhancer directs the expression of a site-specific recombinase in all cells of the transgenic non-human mammal responding to the family of Hedgehog proteins

4. The transgenic non-human animal of either one of claims 2 or 3, wherein the exposure of the non-human transgenic animal to an inducer activates the site-specific recombinase, wherein the activation results in the recombination of two loxP sites, or other site specific recombination target sequences and wherein the recombination of two loxP sites or other site specific recombination target sequences results in genetic marking of a cell and its progeny.

5. The transgenic animal of either one of claims 1 or 2, wherein the reporter gene is selected from the group consisting of β-galactosidase (lacZ), luciferase, a green fluorescent protein, a yellow fluorescent protein, a red fluorescent protein, alkaline phosphatase, or retrograde or anterograde transynaptic tracers such as wheat germ agglutinin (WGA), or derivatives thereof, such as a wheat germ agglutinin-fusion molecule including but not limited to WGA-HRP (wheat germ agglutinin-horseradish peroxidase) or tWGA-dsRED (wheat germ agglutinin-discosoma red), GFP-TTC (green fluorescent protein labeled non-toxic C terminal of tetanus toxin) or any molecule that allows for retrograde and/or anterograde trans-synaptic tracing.

6. A transgenic non-human animal whose genome comprises a transgene comprising a nucleic acid sequence encoding a reporter gene operably linked to a Gli1 promoter that directs expression of the reporter gene in at least one cell of the transgenic animal and its progeny during development and in the adult animal, wherein the reporter gene is only expressed in cells that respond to hedgehog (HH) signaling.

7. A transgenic non-human animal whose genome comprises a transgene comprising a nucleic acid sequence encoding a reporter gene operably linked to a Gli1 promoter that directs expression of the reporter gene in all cells of the transgenic animal and their progeny during development and in the adult animal, wherein the reporter gene is only expressed in cells that respond to hedgehog (HH) signaling.

8. The transgenic animal of any one of claims 1-7, wherein the transgenic non-human animal is used for fate mapping of cells responding to hedgehog signaling in vivo.

9. The transgenic non-human animal of any one of claims 1, 6 or 7, wherein the animal is a rodent selected from the group consisting of a rat, a mouse, a guinea pig and a gerbil.

10. The transgenic non-human animal of any one of claims 1, 6 or 7, wherein the animal is selected from the group consisting of dogs, cats, pigs, sheep, cows, goats, horses, chickens and rabbits.

11. The transgenic non-human animal of claim 1, wherein the site-specific recombinase is selected from the group consisting of CreER, CreER^{T}, CreER^{T2}, CrePR, FlpER, FlpPR, FlpePR, FlpeER, FlpeER^{T,}, FlpeER^{TZ} and derivatives thereof.

12. A method for identifying, marking or following a cell that responds to Hedgehog signaling in vivo comprising the steps of:
a) preparing a transgenic non-human animal whose genome comprises an inducible site-specific recombinase operably linked to a Gli1 promoter enhancer, wherein the promoter directs expression of the recombinase in a cell that responds to Hedgehog proteins, and optionally a reporter gene operably linked to a promoter which directs expression of the reporter gene in at least one cell in the animal, wherein the reporter gene is only expressed in a cell that has a functional recombinase;
b) administering an inducing agent for the site-specific recombinase to the animal in an amount sufficient to induce the recombinase activity, wherein said induction results in activation of the recombinase and subsequent expression of the reporter gene in at least one Hedgehog responding cell of the animal and its progeny; and
c) examining expression of the reporter gene in the cell;
wherein a cell that responds to Hedgehog signaling at the time of administration and its progeny demonstrates expression of the reporter gene.

13. A method for identifying, marking or following cells responding to Hedgehog signaling in vivo comprising the steps of:
a) preparing a transgenic non-human animal whose genome comprises an inducible site-specific recombinase operably linked to a Gli1 promoter enhancer, wherein the promoter directs expression of the recombinase in the cells responding to a Hedgehog protein, and optionally a reporter gene operably linked to a promoter which directs expression of the reporter gene in all cells in the animal that respond to hedgehog signaling, wherein the reporter gene is only expressed in cells that have a functional recombinase;
b) administering an inducing agent for the site-specific recombinase to the animal in an amount sufficient to induce the recombinase activity, wherein said induction results in activation of the recombinase and subsequent expression of the reporter gene in all Hedgehog responding cells of the animal and their progeny; and
c) examining expression of the reporter gene in the cells;
wherein cells that respond to Hedgehog signaling at the time of administration and their progeny demonstrate expression of the reporter gene.

14. The method of either one of claims 12 or 13, wherein the recombinase is selected from the group consisting of CreER, CreER^{T}, CreER^{T2}, CrePR, FlpER, FlpPR, FlpePR, FlpeER, FlpeER^{T,}, FlpeER^{TZ} and derivatives thereof.

15. The method of either one of claims 12 or 13, wherein the inducer is tamoxefin or progesterone.

16. A method for identifying, marking or following a cell that responds to Hedgehog signaling in vivo comprising the steps of:
a) preparing a transgenic non-human animal whose genome comprises a transgene comprising a nucleic acid sequence encoding a reporter gene operably linked to a Gli1 promoter that directs expression of the reporter gene in at least one cell of the transgenic animal and its progeny during development and in the adult animal, wherein the reporter gene is only expressed in a cell that responds to hedgehog (HH) signaling;
b) exposing the transgenic animal to a Hedgehog; and
c) examining expression of the reporter gene in a cell obtained from the animal;
wherein a cell that responds to Hedgehog signaling and its progeny demonstrate expression of the reporter gene.

17. A method for identifying, marking or following cells responding to Hedgehog signaling in vivo comprising the steps of:
a) preparing a transgenic non-human animal whose genome comprises a transgene comprising a nucleic acid sequence encoding a reporter gene operably linked to a Gli1 promoter that directs expression of the reporter gene in all cells of the transgenic animal that respond to hedgehog signaling and their progeny at all times during development and in the adult animal, wherein the reporter gene is only expressed in cells that respond to hedgehog (HH) signaling;
b) exposing the transgenic animal to a Hedgehog; and
c) examining expression of the reporter gene in cells obtained from the animal;
wherein cells that respond to Hedgehog signaling and their progeny demonstrate expression of the reporter gene.

18. The method of any one of claims 12, 13, 16 or 17, wherein the reporter gene is selected from the group consisting of lacZ, a luciferase, a green fluorescent protein, a yellow fluorescent protein, a red fluorescent protein, wheat germ agglutinin (WGA), GFP-TTNC and derivatives thereof.

19. The method of any one of claims 12, 13, 16 or 17, wherein the Hedgehog responding cell is present in mesodermal tissue, epithelial tissue, or neural tissue.

20. The method of any one of claims 12, 13, 16 or 17, wherein the Hedgehog responding cell is present in tissue selected from the group consisting of brain, spinal cord, blood, urogenital, prostate, lung, bladder, kidney, liver, genitalia, eye, lacrimal gland, skin, hair, gut, pancreas and tumor tissue.

21. The method of any one of claims 12, 13, 16 or 17, wherein the cell is selected from the group consisting of an embryonic stem (ES) cell, an adult stem cell, a neural stem cell, an epithelial stem cell, a mesodermal stem cell, a hematopoietic stem cell, a stromal or mesenchymal stem cell, a tumor stem cell, and any cell line derived from the foregoing.

22. The method of claim 21, wherein the neural, epithelial or mesodermal stem cell is a transit amplifying cell or a quiescent stem cell.

23. The method of claim 21, wherein the stromal stem cell becomes a fibroblast or a smooth muscle cell.

24. A method for identifying, marking or following a cell responding to Hedgehog signaling in vitro comprising the steps of:
a) preparing a cell whose genome comprises an inducible CreER^{T2} gene or other site-specific recombinase operably linked to a Gli1 promoter enhancer, wherein the promoter directs expression of the recombinase in a cell that responds to Hedgehog, and a reporter gene operably linked to a promoter which directs expression of the reporter gene in a cell that responds to hedgehog, wherein the reporter gene is only expressed in a cell that has a functional Cre recombinase; or
b) obtaining a cell from an animal whose genome comprises an inducible recombinase gene operably linked to a Gli1 promoter enhancer, wherein the promoter directs expression of the recombinase in any cell responding to a Hedgehog protein, and a reporter gene operably linked to a promoter which directs expression of the reporter gene in a cell from the animal, wherein the reporter is gene is only expressed in a cell that has a functional Cre recombinase;
c) exposing the cell from either of step a) or step b) to an inducing agent in an amount sufficient to induce the recombinase activity, wherein said induction results in expression of the reporter gene in a cell that responds to a Hedgehog protein; and
d) measuring expression of the reporter gene in the cell,
wherein a cell and its progeny that respond to hedgehog signaling demonstrates expression of the reporter gene.

25. The method of claim 24, wherein the inducing agent is tamoxefin or progesterone.

26. The method of claim 24, wherein the reporter gene is selected from the group consisting of lacZ, a luciferase, a green fluorescent protein, a yellow fluorescent protein, a red fluorescent protein, wheat germ agglutinin (WGA), GFP-TTNC and derivatives thereof.

27. The method of claim 24, wherein the cell is isolated from blood, mesodermal tissue, epithelial tissue or neural tissue.

28. The method of claim 24, wherein the cell is isolated from tissue consisting of brain, spinal cord, blood, urogenital, prostate, lung, bladder, kidney, liver, genitalia, eye, lacrimal gland, skin, hair, gut, pancreas and tumor tissue.

29. The method of claim 24, wherein the cell is an embryonic stem (ES) cell, an adult stem cell, a neural stem cell, an epithelial stem, a mesodermal stem cell, a hematopoietic stem cell, a stromal stem cell, a tumor stem cell, and any cell line derived from the foregoing.

30. The method of claim 24, wherein the cell is a quiescent stem cell.

31. The method of claim 30, wherein the quiescent stem cell is a transit amplifying cell.

32. A method of screening for a test compound that acts as an agonist or an antagonist of Hedgehog signaling in vivo, the method comprising the steps of:
a) providing a transgenic non-human animal whose genome comprises either:
i. a transgene comprising a nucleic acid sequence encoding an inducible recombinase gene operably linked to a Gli1 promoter enhancer and a reporter gene, activated by the recombinase, wherein the promoter directs expression of the recombinase and the reporter gene in a cell that responds to Hedgehog signaling in the non-human transgenic animal upon exposure of the animal to an inducing agent; or
ii. a transgene comprising a nucleic acid sequence encoding a reporter gene operably linked to a Gli1 promoter that directs expression of the reporter gene in a cell of the transgenic animal and its progeny that respond to hedgehog signaling during development and in the adult animal, wherein the reporter gene is only expressed in a cell that responds to hedgehog (HH) signaling;
b) administering a test compound to the animal of step a) i or a) ii;
c) exposing the animal to an inducing agent in an amount sufficient to induce recombinase activity if the animal from step a) i is used;
d) observing for a cell and its progeny that expresses the reporter gene; and
e) comparing the level of expression of the reporter gene in animals treated with the test compound to the level of expression of the reporter gene expressed in the transgenic animals not exposed to the test compound,
wherein a test compound is identified as an agonist or antagonist of Hedgehog signaling if the level of expression of the reporter gene is changed in animals administered the test compound compared to the level of expression of the reporter gene in animals not receiving the test compound.

33. The method of claim 32, wherein the reporter gene is selected from the group consisting of β-galactosidase (lacZ), luciferase, a green fluorescent protein, a yellow fluorescent protein, a red fluorescent protein, alkaline phosphatase, or retrograde or anterograde transynaptic tracers such as wheat germ agglutinin (WGA), or derivatives thereof, such as a wheat germ agglutinin-fusion molecule including but not limited to WGA-HRP (wheat germ agglutinin-horseradish peroxidase) or tWGA-dsRED (wheat germ agglutinin-discosoma red), GFP-TTC (green fluorescent protein labeled non-toxic C terminal of tetanus toxin) or any molecule that allows for retrograde and/or anterograde trans-synaptic tracing.

34. The method of claim 32, wherein the test compound is identified as an antagonist of Hedgehog signaling if the level of expression of the reporter gene is lower in animals administered the test compound compared to the level of expression of the reporter gene in animals not receiving the test compound.

35. The method of claim 32, wherein the test compound is identified as an agonist of Hedgehog signaling if it induces higher expression of the reporter gene in new (not pre-existing cells) cells incubated with the test compound compared to the expression of the reporter gene in mammals not receiving the test compound.

36. A method of screening for a test compound that acts as an agonist or an antagonist of Hedgehog signaling in vitro, the method comprising the steps of:
a) preparing a hedgehog responsive cell whose genome comprises either:
i. a nucleic acid sequence encoding a Gli1 gene, an inducible recombinase gene and a reporter gene activated by the recombinase, all of which are operably linked to a Gli1 promoter enhancer, wherein the promoter directs expression of Gli1, the recombinase and the reporter gene upon exposure of the cell to an inducing agent; or
ii. a nucleic acid sequence encoding a reporter gene operably linked to a Gli1 promoter that directs expression of the reporter gene in the cell, wherein the reporter gene is only expressed in a cell that responds to hedgehog (HH) signaling;
b) incubating the cell of step a) i or a) ii. with a test compound;
c) exposing the cell to an inducing agent if the cell of step a) i is used;
d) measuring expression of the reporter gene in the cell and its progeny; and
e) comparing the level of expression of the reporter gene in the cell treated with the test compound to the level of expression of the reporter gene in the cell not exposed to the test compound,
wherein a test compound is identified as an agonist or antagonist of Hedgehog signaling if the level of expression of the reporter gene is changed in the cell treated with the test compound compared to the level of expression of the reporter gene in the cell not treated with the test compound.

37. The method of claim 36, wherein a test compound is identified as an antagonist of Hedgehog signaling if the level of expression of the reporter gene is lower in the cell incubated with the test compound compared to the level of expression of the reporter gene in the cell not incubated with the test compound.

38. The method of claim 36, wherein the test compound is identified as an agonist of Hedgehog signaling if the expression of the reporter gene is greater in cells incubated with the test compound compared to the expression of the reporter gene in a cell not incubated with the test compound.

39. The method of claim 36, wherein the reporter gene is selected from the group consisting of lacZ, a luciferase, a green fluorescent protein, a red fluorescent protein, a yellow fluorescent protein, wheat germ agglutinin (WGA), a WGA type molecule, GFP-TTNC and derivatives therof.
